(19) United States
(12) Patent Application Publication
(10) Pub. No.: US 2023/0149366 A1
(43) Pub. Date: May 18, 2023

(54) SOLID DOSAGE FORMS OF TAFAMIDIS

(71) Applicant: Nuray Chemicals Private Limited, Thiruvallur, Tamilnadu (IN)

(72) Inventors: Muthusamy SHANMUGAM, East Windsor, NJ (US); Singavarapu Ajay MADHUKAR, Thiruvallur (IN); Palanisamy SIVAKUMAR, Thirumazhisai (IN); Thirunavukkarasu SAKTHIKUMAR, Thirumazhisai (IN)

(73) Assignee: Nuray Chemicals Private Limited, Thiruvallur, Tamilnadu (IN)

(21) Appl. No.: 18/054,185

(22) Filed: Nov. 10, 2022

Related U.S. Application Data
(63) Continuation-in-part of application No. 17/530,003, filed on Nov. 18, 2021, now Pat. No. 11,523,993.

(51) Int. Cl. A61K 31/423; A61K 9/00; A61K 9/48; A61K 9/20

(52) U.S. Cl. CPC A61K 31/423; A61K 9/0053; A61K 9/4858; A61K 9/4866; A61K 9/4825; A61K 9/485; A61K 9/2018; A61K 9/2013; A61K 9/2054; A61K 9/2027; A61K 9/2009

(57) ABSTRACT

The disclosure relates to a dosage form comprising a hard gelatin or a HPMC capsule having a granule composition comprising tafamidis or its pharmaceutically acceptable salt particularly tafamidis meglumine or solid-state forms or polymorphic forms of tafamidis particularly a solid-state form comprising tafamidis and fumaric acid. The disclosure also relates to a tablet comprising tafamidis or its pharmaceutically acceptable salt particularly tafamidis meglumine or solid-state forms or polymorphic forms of tafamidis particularly a solid-state form comprising tafamidis and fumaric acid. The solid dosage forms disclosed herein do not form a rigid gel upon contacting with water or buffer solution in dissolution specifically pH 6.8 phosphate buffer and the compositions disclosed herein are indicated for the treatment of the cardiomyopathy of wild type or hereditary transthyretin-mediated amyloidosis in adults to reduce cardiovascular mortality and cardiovascular-related hospitalization.

### RELATED APPLICATION

This application is a continuation-in-part of U.S. patent application Ser. No. 17/530,003, filed on Nov. 18, 2021.

### FIELD OF THE INVENTION

The disclosure relates to a dosage form comprising tafamidis or a pharmaceutically acceptable salt thereof in the form of a hard gelatin capsule or tablet comprising a granule composition comprising tafamidis or a pharmaceutically acceptable salt thereof (e.g., tafamidis meglumine) that would not form a rigid gel upon contacting with water or buffer solution in dissolution specifically pH 6.8 phosphate buffer saline (PBS), wherein said compositions are indicated for the treatment of the cardiomyopathy of wild type or hereditary transthyretin-mediated amyloidosis in adults to reduce cardiovascular mortality and cardiovascular-related hospitalization.

### BACKGROUND

Tafamidis is a selective stabilizer of transthyretin. Tafamidis is chemically known as 2-(3,5-dichlorophenyl)-1,3-benzoxazole-6-carboxylic acid with a molecular weight of 308.12 g/mol. Tafamidis meglumine is chemically known as 2-(3,5-dichlorophenyl)-1,3-benzoxazole-6-carboxylic acid mono (1-deoxy-1-methylamino-D-glucitol) with a molecular weight of 503.33 g/mol.

Tafamidis and tafamidis meglumine are available as soft gelatin capsule form in United States, Europe, and other markets. Tafamidis is marketed as Vyndamax®, while tafamidis meglumine is marketed as Vyndaqel®. The Vyndaqel® European Public assessment report states that “[d]uring development it was shown that tafamidis active substance, when formulated in a hard gelatin capsule with standard excipients, formed a rigid gel structure upon contact with aqueous vehicles thereby affecting the dissolution rate from the solid dosage.” The Vyndaqel® European Public assessment report also states that “[b]ased on the initial dissolution results, it was decided to further enhance drug dispersibility and the dissolution rate using a soft gelatin capsule dosage form,” and that “[b]ased on this the strategy was to prepare a water-dispersible drug suspension that would not form a gel upon contacting with water and encapsulate the suspension in a soft gelatin capsule.” The Vyndaqel® European Public assessment report further states that “[a] suspension formulation was required as tafamidis showed poor solubility in many of the vehicles commonly used in formulating soft gelatin capsules and thus eliminated the option of developing the drug in a solution form,” and that “selection and optimization of individual components of the proposed commercial formulation were discussed in detail.”

According to the Vyndaqel® European Public assessment report “[t]he chosen excipients in the capsule fill solution are: polyethylene glycol (suspending vehicle), sorbitan monoleate (surfactant/wetting agent), polysorbate 80 (surfactant/wetting agent),” while “[t] he excipients in the capsule shell are: gelatin, propylene glycol, purified water, ‘Sorbitol special-glycerin blend’ (d-sorbitol, 1,4 sorbitan, mannitol and glycerin) and titanium dioxide (E 171) and black printing ink.”

WO2021001858A1, WO2021019448A1, and WO2020232325A1, describe processes for the preparation of various polymorphic forms of tafamidis and tafamidis meglumine including an amorphous premix of tafamidis meglumine and spray drying of tafamidis meglumine to obtain amorphous tafamidis meglumine. These patent documents generally describe pharmaceutical compositions for tafamidis and its pharmaceutically acceptable salt, but are silent about manufacturing of particular composition.

WO2020128816A2 describes different conventional formulations of tafamidis and tafamidis meglumine without any information related to the stability challenges associated with a tafamidis-containing formulation.

During extensive investigations, it was discovered that tafamidis-containing dosage forms (e.g., hard-shell capsules and tablets) having acceptable drug dispersibility and dissolution rates may be obtained without using “special” ingredients. The inventors surprisingly found that the formation of rigid gels may be circumvented based on the formulated compositions disclosed herein.

### Object

The main objective of the disclosure relates to a pharmaceutically acceptable composition, preferably hard gelatin capsules, comprising tafamidis or tafamidis meglumine or its solid-state forms or polymorphic forms thereof and one or more excipients, wherein formulated in a hard gelatin capsule with standard excipients, having easily water-dispersible carrier that would not form a rigid gel upon contacting water or buffer solution in dissolution a specifically in an official media of pH 6.8 phosphate buffer.

Another objective of the present disclosure relates to pharmaceutically acceptable tablet composition comprising standard excipients, having easily water-dispersible carrier that would not form a rigid gel upon contacting water or buffer solution in dissolution a specifically in an official media of pH 6.8 phosphate buffer.

### SUMMARY

In present disclosure provides a hard gelatin capsule composition having easily dispersible granules for oral administration comprising:
a) Tafamidis or its pharmaceutically acceptable salt or its solid-state forms or polymorphic forms thereof;
b) at least one acidifier; and
c) at least one pharmaceutically acceptable excipient selected from the group consisting of at least one surfactant; disintegrant, binder, diluent, glidant and lubricant
   Or
      A hard gelatin capsule composition having spray-dried or evaporated composition for oral administration comprising:
a) Tafamidis or its pharmaceutically acceptable salt or its solid-state forms or polymorphic forms thereof;
b) at least one solubility enhancer;
c) at least one disintegrant; and
d) at least one pharmaceutically acceptable excipient.
   In another aspect of the present disclosure is related to novel oral tablet composition comprising;
a) Tafamidis or its pharmaceutically acceptable salt or its solid-state forms or polymorphic forms thereof;
b) at least one acidifier; and
c) at least one pharmaceutically acceptable excipient selected from the group consisting of at least one surfactant; disintegrant, binder, diluent, glidant and lubricant.
   In another aspect of the present disclosure is related to solid oral dosage form comprising;
a) tafamidis or its pharmaceutically acceptable salt or its solid-state forms or polymorphic forms thereof;
b) at least one acidifier in an amount of about 0% w/w to about 10% w/w based on the total weight of the dosage form; and
c) at least one pharmaceutically acceptable excipient selected from the group consisting of at least one disintegrant, binder, diluent, glidant and lubricant; wherein the present composition does not form a gel during dissolution in a suitable medium.

### BRIEF DESCRIPTION OF THE DRAWINGS

FIG. 1: Formation of a rigid gel in hard gelatin capsule prepared as per example-1 containing tafamidis meglumine granules in pH 6.8 Phosphate buffer at 30 minutes (Q point).

FIG. 2: Formation of a rigid gel in hard gelatin capsule prepared as per example-2 containing tafamidis meglumine granules in pH 6.8 Phosphate buffer at 30 minutes (Q point).

FIG. 3: Formation of gel in hard gelatin capsule prepared as per example-3 containing tafamidis meglumine granules in pH 6.8 Phosphate buffer at 30 minutes (Q point).

FIG. 4: Formation of gel in hard gelatin capsule prepared as per example-4 containing tafamidis Meglumine granules in pH 6.8 Phosphate buffer at 30 minutes (Q point).

FIG. 5: No gel/gel structured mucilage formation in pH 6.8 Phosphate buffer at 30 minutes (Q point) of hard gelatin capsule prepared as per Example-6 containing tafamidis meglumine granules with organic acid i.e. citric acid anhydrous.

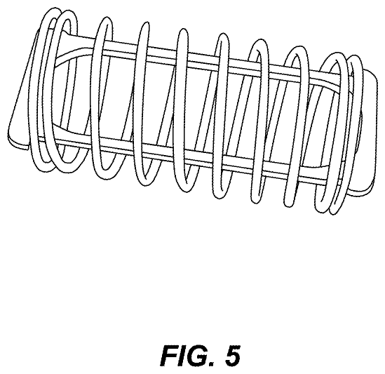

### DETAILED DESCRIPTION

The information that follows illustrates various embodiments of the compositions disclosed herein. For the avoidance of doubt, it is specifically intended that any particular feature(s) described individually in any one of these paragraphs (or part thereof) may be combined with one or more other features described in one or more of the remaining paragraphs (or part thereof). In other words, it is explicitly intended that the features described below individually in each paragraph (or part thereof) represent important aspects of the invention that may be taken in isolation and also combined with other important aspects of the invention described elsewhere within this specification as a whole, and including the examples and figures. The skilled person will appreciate that the compositions claimed herein extends to such combinations of features and that these have not been recited in detail here in the interests of brevity.

Definitions of some of the terms used herein are detailed below.

The use of the terms “a” and “an” and “the” and similar references in the context of describing the liquid composition described herein (especially in the context of the following claims) are to be construed to cover both the singular and the plural, unless otherwise indicated herein or clearly contradicted by context.

The term “about” as used herein embodies standard error associated with a physico-chemical observable. As used herein, the term “about” means a slight variation of the value specified, for example, within 10% of the value specified. A stated amount for a compositional ingredient that is not preceded by the term “about” does not mean that there is no variance for the stated term, as one of ordinary skill would understand that there may be the possibility of a degree of variability generally associated with experimental error.

The expression “TFASSF,” as used herein relates to a solid-state form comprising tafamidis and fumaric acid.

The term “therapeutically effective amount” or effective dose” as used herein refers to the amount or dose of tafamidis, tafamidis meglumine, or TFASSF that is sufficient to initiate therapeutic response in a patient.

A first embodiment relates to hard gelatin capsule composition oral administration comprising: easily dispersible granules comprising a) tafamidis or a pharmaceutically acceptable salt thereof or its solid-state forms or polymorphic forms thereof; b) at least one acidifier; and c) at least one pharmaceutically acceptable excipient comprising a surfactant, a disintegrant, a binder, a diluent, a glidant, a lubricant, or a combination thereof.

A second embodiment relates to a hard gelatin capsule composition oral administration comprising spray-dried or evaporated composition comprising a) tafamidis or its pharmaceutically acceptable salt or its solid-state forms or polymorphic forms thereof; b) at least one solubility enhancer; c) at least one disintegrant; and d) at least one pharmaceutically acceptable excipient.

In one aspect of the first or second embodiment, the hard gelatin capsule composition for oral administration comprises about 20 mg of tafamidis meglumine.

In another aspect of the first or second embodiment, the hard gelatin capsule composition for oral administration comprises about 61 mg of tafamidis.

In another aspect of the first or second embodiment, the hard gelatin capsule composition for oral administration comprises TFASSF comprising about 61 mg of tafamidis.

As related to TFASSF, it may be convenient to express the amount of fumaric acid in TFASSF as the weight-percent of fumaric acid based on the total weight of TFASSF, which may range: (i) from about 14% w/w to about 18% w/w based on the total weight of the solid-state form (“TFASSF(14-18)”), (ii) from about 15% w/w to about 17% w/w based on the total weight of the solid-state form (“TFASSF(15-17)”), or (iii) from about 15.9% w/w to about 16.2% w/w based on the total weight of the solid-state form. Alternatively, TFASSF may comprise fumaric acid in an amount of about 16% w/w based on the total weight of the solid-state form (TFASSF(16)).

In one aspect of the first embodiment, the granules comprise tafamidis or a pharmaceutically acceptable salt thereof or its solid-state forms or polymorphic forms thereof, such as, for example, tafamidis meglumine, and the tafamidis solid-state form comprising tafamidis and fumaric acid in an amount of from about 14% w/w to about 17% w/w based on the total weight of the solid-state form.

In another aspect of the first embodiment, the granules comprise tafamidis meglumine in an amount of about 20 mg per capsule.

In another aspect of the first embodiment, the granules comprise tafamidis in an amount of about 61 mg per capsule.

In another aspect of the first embodiment, the granules comprise about 72.5 mg per capsule of TFASSF, e.g., the tafamidis solid-state form comprising tafamidis and fumaric acid in an amount of from about 14% w/w to about 17% w/w based on the total weight of the solid-state form.

In another aspect of the second embodiment, the spray dried/evaporated solid composition comprises tafamidis or a pharmaceutically acceptable salt or its solid-state forms or polymorphic forms thereof. In another aspect of the second embodiment, the spray dried/evaporated solid composition comprises tafamidis meglumine in an amount of about 20 mg per capsule.

In another aspect of the second embodiment, the spray dried/evaporated solid composition comprises tafamidis in an amount of about 61 mg per capsule.

In another aspect of the second embodiment, the spray dried/evaporated solid composition comprises about 61 mg tafamidis (as, e.g., TFASSF(16)) per capsule.

A third embodiment relates to a tablet composition comprising tafamidis or a pharmaceutically acceptable salt or its solid-state forms or polymorphic forms thereof; b) at least one acidifier; and c) at least one pharmaceutically acceptable excipient comprising a surfactant, a disintegrant, a binder, a diluent, a glidant, a lubricant, or a combination thereof.

In one aspect of the third embodiment, the tablet comprises tafamidis meglumine in an amount of about 20 mg per tablet.

In another aspect of the third embodiment, the tablet comprises tafamidis in an amount of about 61 mg per tablet.

In another aspect of the third embodiment, the tablet comprises about 61 mg tafamidis (as, e.g., TFASSF(16)) per tablet.

In one aspect of an embodiment disclosed herein, the composition may comprise at least one acidifier which may be a pharmaceutically acceptable organic acid, inorganic acid, or a combination thereof. Examples of pharmaceutically acceptable organic acids and inorganic acids include, but are not limited to hydrochloric acid, hydrobromic acid, hydroiodic acid, sulfuric acid, sulfamic acid, phosphoric acid, nitric acid, citric acid, tartaric acid, malic acid, fumaric acid, salicylic acid, p-toluenesulfonic acid, methanesulfonic acid, oxalic acid, succinic acid, lactic acid, or a combination thereof. As explained in greater detail herein, an unexpected discovery regarding the development work disclosed herein is that an acidifier inhibits gel formation during the dissolution. In one aspect, the acidifier comprises hydrochloric acid, phosphoric acid, citric acid, tartaric acid, malic acid, fumaric acid, succinic acid, lactic acid, or a combination thereof.

In one aspect of an embodiment disclosed herein, the at least one acidifier may be present in an amount of from about 0% w/w to about 10% w/w based on the total weight of the composition, including all values in between, such as, for example, about 0.1% w/w, about 0.2% w/v, about 0.3% w/w, about 0.4% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, and about 9.5% w/w.

In yet another aspect of an embodiment disclosed herein, the composition may comprise at least one surfactant. Examples of surfactants include but are not limited to sodium lauryl sulfate, poloxamer, glyceryl monostearate, glyceryl monolaurate, sorbitan monolaurate, sorbitan monostearate, polyethylene glycols, or a combination thereof. Observations made during development work shows that the at least one surfactant may increase in vivo bioavailability upon oral administration of a composition disclosed herein.

In one aspect of an embodiment disclosed herein, the at least one surfactant may be present in an amount of from about 0.1% w/w to about 10% w/w based on the total weight of the composition, including all values in between, such as, for example, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, and about 9.5% w/w.

In one aspect of an embodiment disclosed herein, the composition may comprise at least one disintegrant. Examples of disintegrant include, but are not limited to, crospovidone, croscarmellose sodium, low hydroxypropyl cellulose, starch, sodium starch glycolate, microcrystalline cellulose, alginic acid, polacrillin potassium, or a combination thereof.

In one aspect of an embodiment disclosed herein, the at least one disintegrant may be present in an amount of from about 0% w/w to about 10% w/w based on the total weight of the composition, including all values in between, such as, for example, about 0.05% w/w, about 0.1% w/w, about 0.2% w/w, about 0.3% w/w, about 0.4% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, and about 9.5% w/w. In one aspect of an embodiment disclosed herein, the composition may comprise at least one binder. Examples of binders include but are not limited to povidone, starch, gelatin, hydroxypropyl cellulose, hydroxypropyl methylcellulose, methyl cellulose, carboxymethyl cellulose, or a combination thereof.

In one aspect of an embodiment disclosed herein, the at least one binder may be present in an amount of from about 0% w/w to about 10% w/w based on the total weight of the composition, including all values in between, such as, for example, about 0.05% w/w, about 0.1% w/w, about 0.2% w/w, about 0.3% w/w, about 0.4% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, and about 9.5% w/w.

In one aspect of an embodiment disclosed herein, the composition may comprise at least one diluent. Examples of diluents include, but are not limited to lactose, microcrystalline cellulose, starch, dicalcium phosphate, mannitol, xylitol, sorbitol, dextrose, fructose, sucrose, maltodextrin, or a combination thereof.

In one aspect of an embodiment disclosed herein, the at least one diluent may be present in an amount of from about 0% w/w to about 90% w/w based on the total weight of the composition, including all values in between, such as, for example, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, about 9.5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, and about 85% w/w.

In one aspect of an embodiment disclosed herein, the composition may comprise at least one solubility enhancer. Examples of solubility enhancers include, but are not limited to, α-cyclodextrin, γ-cyclodextrin, β-cyclodextrin (and derivatives thereof; see, e.g., U.S. Patent Application Publication No. 2014/0377185 A9), hydroxypropyl-J3-cyclodextrin, povidone, copovidone, polyethylene glycol, sorbitol monooleate polysorbate, or a combination thereof.

In one aspect of an embodiment disclosed herein, the at least one solubility enhancer may be present in an amount of from about 0% w/w to about 90% w/w based on the total weight of the composition, including all values in between, such as, for example, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, about 9.5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, and about 85% w/w.

In one aspect of an embodiment disclosed herein, the composition may comprise at least one glidant. Examples of glidants include, but are not limited to, colloidal silicon dioxide, magnesium trisilicate, starch, talc, or a combination thereof.

In one aspect of an embodiment disclosed herein, the at least one glidant may be present in an amount of from about 0% w/w to about 2% w/w based on the total weight of the composition, including all values in between, such as, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, and about 1.9% w/w.

In one aspect of an embodiment disclosed herein, the composition may comprise at least one lubricant. Examples of lubricants include, but are not limited to, calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, medium-chain triglycerides, mineral oil, myristic acid, palmitic acid, poloxamer, polyethylene glycol, sodium stearyl fumarate, stearic acid, talc, zinc stearate, or a combination thereof.

In one aspect of an embodiment disclosed herein, the at least one lubricant may be present in an amount of from about 0% w/w to about 2% w/w based on the total weight of the composition, including all values in between, such as, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w, and about 1.9% w/w.

A fourth embodiment relates to a solid oral dosage form comprising tafamidis or a pharmaceutically acceptable salt or its solid-state forms or polymorphic forms thereof; and at least one pharmaceutically acceptable excipient comprising a disintegrant, a binder, a diluent, a glidant, and a lubricant, or a combination thereof.

In another aspect of the fourth embodiment, the solid oral dosage form comprises a hard gelatin capsule, a HPMC capsule, or a tablet.

In another aspect of the fourth embodiment, the solid oral dosage form comprises tafamidis meglumine in an amount of about 20 mg per capsule or tablet.

In another aspect of the fourth embodiment, the solid oral dosage form comprises tafamidis in an amount of about 61 mg per capsule or about 12.2 mg per capsule or tablet.

In another aspect of the fourth embodiment, the solid oral dosage form comprises about 61 mg tafamidis in the form of TFASSF (as, e.g., TFASSF(16)) in an per capsule or tablet.

In one aspect of an embodiment disclosed herein (e.g., the fourth embodiment), the composition may comprise at least one disintegrant. Examples of disintegrant include, but are not limited to, crospovidone, croscarmellose sodium, low hydroxypropyl cellulose, starch, sodium starch glycolate, microcrystalline cellulose, alginic acid, polacrillin potassium, or a combination thereof.

In one aspect of an embodiment disclosed herein (e.g., the fourth embodiment), the at least one disintegrant may be present in an amount of from about 0% w/w to about 10% w/w based on the total weight of the composition, including all values in between, such as, for example, about 0.05% w/w, about 0.1% w/w, about 0.2% w/w, about 0.3% w/w, about 0.4% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w and about 9.5% w/w.

In yet another aspect of an embodiment disclosed herein (e.g., the fourth embodiment), the composition optionally comprises at least one surfactant. Examples of surfactants include but are not limited to sodium lauryl sulfate, poloxamer, glyceryl monostearate, glyceryl monolaurate, sorbitan monolaurate, sorbitan monostearate, polyethylene glycols, or a combination thereof. Observations made during development work shows that the at least one surfactant may increase in vivo bioavailability upon oral administration of a composition disclosed herein.

In yet another aspect of an embodiment disclosed herein (e.g., the fourth embodiment), the composition does not include a surfactant, such as an anionic surfactant, a cationic surfactant, or a non-ionic surfactant, such as a polysorbate (e.g., polysorbate 20, polysorbate 40, polysorbate 60, polysorbate 80, and the like).

In one aspect of an embodiment disclosed herein (e.g., the fourth embodiment), the composition optionally comprises at least one surfactant may be present in an amount of from about 0.1% w/w to about 10% w/w based on the total weight of the composition, including all values in between, such as, for example, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, and about 9.5% w/w.

In one aspect of an embodiment disclosed herein (e.g., the fourth embodiment), the composition may comprise at least one binder. Examples of binders include but are not limited to povidone, starch, gelatin, hydroxypropyl cellulose, hydroxypropyl methylcellulose, methyl cellulose, carboxymethyl cellulose, or a combination thereof.

In one aspect of an embodiment disclosed herein (e.g., the fourth embodiment), the at least one binder may be present in an amount of from about 0% w/w to about 10% w/w based on the total weight of the composition, including all values in between, such as, for example, about 0.05% w/w, about 0.1% w/w, about 0.2% w/w, about 0.3% w/w, about 0.4% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, and about 9.5% w/w.

In one aspect of an embodiment disclosed herein (e.g., the fourth embodiment), the composition may comprise at least one diluent. Examples of diluents include, but are not limited to lactose, microcrystalline cellulose, starch, dicalcium phosphate, mannitol, xylitol, sorbitol, dextrose, fructose, sucrose, maltodextrin, or a combination thereof.

In one aspect of an embodiment disclosed herein (e.g., the fourth embodiment), the at least one diluent may be present in an amount of from about 0% w/w to about 90% w/w based on the total weight of the composition, including all values in between, such as, for example, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, about 9.5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, and about 85% w/w.

In one aspect of an embodiment disclosed herein (e.g., the fourth embodiment), the composition may comprise at least one solubility enhancer. Examples of solubility enhancers include, but are not limited to, α-cyclodextrin, γ-cyclodextrin, β-cyclodextrin (and derivatives thereof; see, e.g., U.S. Patent Application Publication No. 2014/0377185 A9), hydroxypropyl-β-cyclodextrin, povidone, copovidone, polyethylene glycol, sorbitol monooleate polysorbate, or a combination thereof.

In one aspect of an embodiment disclosed herein (e.g., the fourth embodiment), the at least one solubility enhancer may be present in an amount of from about 0% w/w to about 90% w/w based on the total weight of the composition, including all values in between, such as, for example, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.5% w/w, about 7.0% w/w, about 7.5% w/w, about 8.5% w/w, about 9.0% w/w, about 9.5% w/w, about 10% w/w, about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, and about 85% w/w.

In one aspect of an embodiment disclosed herein (e.g., the fourth embodiment), the composition may comprise at least one glidant. Examples of glidants include, but are not limited to, colloidal silicon dioxide, magnesium trisilicate, starch, talc, or a combination thereof.

In one aspect of an embodiment disclosed herein (e.g., the fourth embodiment), the at least one glidant may be present in an amount of from about 0% w/w to about 2% w/w based on the total weight of the composition, including all values in between, such as, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w and about 1.9% w/w.

In one aspect of an embodiment disclosed herein (e.g., the fourth embodiment), the composition may comprise at least one lubricant. Examples of lubricants include, but are not limited to, calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, medium-chain triglycerides, mineral oil, myristic acid, palmitic acid, poloxamer, polyethylene glycol, sodium stearyl fumarate, stearic acid, talc, zinc stearate, or a combination thereof.

In one aspect of an embodiment disclosed herein (e.g., the fourth embodiment), the at least one lubricant may be present in an amount of from about 0% w/w to about 2% w/w based on the total weight of the composition, including all values in between, such as, for example, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.1% w/w, about 1.2% w/w, about 1.3% w/w, about 1.4% w/w, about 1.5% w/w, about 1.6% w/w, about 1.7% w/w, about 1.8% w/w and about 1.9% w/w.

In one aspect of an embodiment disclosed herein (e.g., the fourth embodiment), the solid oral dosage form, comprising: a) a solid-state form comprising tafamidis and fumaric acid (e.g., TFASSF) b) at least one acidifier in an amount of about 0% w/w to about 10% w/w based on the total weight of the dosage form and c) at least one pharmaceutically acceptable excipient; wherein the solid-state form comprises fumaric acid in an amount of from about 14% w/w to about 18% w/w based on the total weight of the solid-state form.

In one aspect of an embodiment disclosed herein (e.g., the fourth embodiment), the solid oral dosage form, comprising: a) a solid-state form comprising about 61 mg tafamidis and fumaric acid (e.g., TFASSF) b) at least one acidifier in an amount of about 0% w/w to about 10% w/w based on the total weight of the dosage form and c) at least one pharmaceutically acceptable excipient; wherein the solid-state form comprises fumaric acid in an amount of from about 14% w/w to about 18% w/w based on the total weight of the solid-state form.

In one aspect of an embodiment disclosed herein (e.g., the fourth embodiment), the solid-state form comprises tafamidis and fumaric acid in an amount of from about 15% w/w to about 17% w/w based on the total weight of the solid-state form.

In one aspect of an embodiment disclosed herein (e.g., the fourth embodiment), the solid-state form comprises about 61 mg tafamidis and fumaric acid in an amount of from about 15% w/w to about 17% w/w based on the total weight of the solid-state form.

In one aspect of an embodiment disclosed herein (e.g., the fourth embodiment), the solid-state form comprises tafamidis and fumaric acid in an amount of from about 15.9% w/w to about 16.2% w/w based on the total weight of the solid-state form.

In one aspect of an embodiment disclosed herein (e.g., the fourth embodiment), the solid-state form comprises about 61 mg tafamidis and fumaric acid in an amount of from about 15.9% w/w to about 16.2% w/w based on the total weight of the solid-state form.

In one aspect of an embodiment disclosed herein (e.g., the fourth embodiment), the solid-state form comprises tafamidis and fumaric acid in an amount of about 16% w/w based on the total weight of the solid-state form.

In one aspect of an embodiment disclosed herein (e.g., the fourth embodiment), the solid-state form comprises about 61 mg tafamidis and fumaric acid in an amount of about 16% w/w based on the total weight of the solid-state form.

In one aspect of an embodiment disclosed herein (e.g., the fourth embodiment), the lower particle size of tafamidis or its pharmaceutically acceptable salt and its solid-state forms thereof (e.g., D90 less than about 15 μm) exhibit fast release during the administration. The solid oral dosage form manufactured by using tafamidis or its pharmaceutically acceptable salt and its solid-state forms having a lower D90 particle size of less than 15 μm (or a D90 less than 15 μm) released tafamidis faster than the reference product. It was discovered that a solid oral dosage form manufactured using a tafamidis form (e.g., salt, solid-state, polymorphic) having particle size of about 20 microns (or greater) released the tafamidis at a rate comparable to the reference product (e.g., Vyndamax). Accordingly, a suitable D90 particle size for the tafamidis form (e.g., salt, solid-state, polymorphic) disclosed herein ranges from about 20 μm to about 100 μm, and all values in between including, for example about 21 μm, about 22 μm, about 23 μm, about 24 μm, about 25 μm μm, 26 μm, about 27 μm, about 28 μm, about 29 μm, about 30 μm, about 31 μm, about 32 μm, about 33 μm, about 34 μm, about 35 μm μm, about 36 μm, about 37 μm, about 38 μm, about 39 μm, about 40 μm, about 41 μm, about 42 μm, about 43 μm, about 44 μm, about 45 μm μm, about 46 μm, about 47 μm, about 48 μm, about 49 μm, about 50 μm, about 51 μm, about 52 μm, about 53 μm, about 54 μm, about 55 μm μm, about 56 μm, about 57 μm, about 58 μm, about 59 μm, about 60 μm, about 61 μm, about 62 μm, about 63 μm, about 64 μm, about 65 μm μm, about 66 μm, about 67 μm, about 68 μm, about 69 μm, about 70 μm, about 71 μm, about 72 μm, about 73 μm, about 74 μm, about 75 μm μm, about 76 μm, about 77 μm, about 78 μm, about 79 μm, about 80 μm, about 81 μm, about 82 μm, about 83 μm, about 84 μm, about 85 μm μm, about 86 μm, about 87 μm, about 88 μm, about 89 μm, about 90 μm, about 91 μm, about 92 μm, about 93 μm, about 94 μm, about 95 μm μm, about 96 μm, about 97 μm, about 98 μm, and about 99 μm.

An aspect of an embodiment disclosed herein (e.g., the fourth embodiment) relates to a method of treating transthyretin amyloid disease in a mammal, comprising administering to the mammal the solid oral dosage form of the embodiment disclosed herein (e.g., the fourth embodiment).

Another aspect relates to a method of treating transthyretin amyloid disease in a mammal, comprising administering to the mammal the solid oral dosage form of the embodiment disclosed herein (e.g., the fourth embodiment, e.g., a solid dosage form comprising about 72.5 mg of TFASSF(16) having a D90 of about 20 μm to about 100 μm)) wherein an observed pharmacokinetic parameter (e.g., Cmax and/or AUC0-72) is substantially the same as a reference product (e.g., Vyndamax), where the observed pharmacokinetic parameter includes, but is not limited to a Cmax-value of from about 3.5 ng/mL to about 5.5 ng/mL (e.g., about 4.4 ng/mL, about 4.5 ng/mL, or about 4.7 ng/mL), an AUC0-72-value of from about 120 hr·ng/mL to about 195 hr·ng/mL (e.g., about 154 hr·ng/mL, about 152 hr·ng/mL, or about 170 hr·ng/mL).

In one aspect of an embodiment disclosed herein, the capsule composition or the tablet composition comprising the readily available premix composition of two or more pharmaceutically acceptable excipients or readily available premix composition of two or more pharmaceutically acceptable excipients with or without combination of acidifier.

In one aspect of an embodiment disclosed herein, the capsule composition or the tablet composition does not form a gel during dissolution in a suitable medium.

Capsule compositions disclosed herein comprise a hard capsule having a capsule size suitable based on the total amount of the capsule composition. Examples of suitable capsule sizes include, for example, #0, #1, #2, #3, and #4.

### EXAMPLES

The following exemplified embodiments illustrate aspects of the solid composition disclosed herein and should not be considered to be limiting on the subject matter claimed herein.

The FDA recommends two dissolution methods for tafamidis and tafamidis meglumine.

The dissolution method for tafamidis (capsule) utilizes a USP II (Paddle) apparatus with sinker operating at a speed of 75 rpm and a medium volume of 900 mL, where the Tier I Medium comprises 0.05 M (50 mM) Sodium Phosphate Buffer, pH 6.8 with 1.0% Tween 80 and the Tier II Medium comprises 0.05 M (50 mM) Sodium Phosphate Buffer, pH 6.8 with 1.0% Tween 80 and enzyme with protease activity as per USP. The recommended sampling times are 5, 10, 15, 20, 30, and 45 minutes.

The dissolution method for tafamidis meglumine (capsule) utilizes a USP II (Paddle) apparatus with sinker operating at a speed of 75 rpm and a medium volume of 900 mL, where the Tier 1 Medium comprises 0.05 M (50 mM) Sodium Phosphate Buffer, pH 6.8, and the Tier 2 Medium comprises 0.05 M (50 mM) Sodium Phosphate Buffer, pH 6.8, and pancreatin with protease activity as per USP. The recommended sampling times are 5, 10, 15, 20, 30, and 45 minutes.

The FDA recommended dissolution medium disclosed herein may be referred to as the “official media” or “official medium (OGD).”

TFASSF comprising tafamidis and fumaric acid in an amount of from about 14% w/w to about 18% w/w (e.g., about 16% fumaric acid, viz., TFASSF(16)) based on the total weight of the solid-state form may be prepared by combining a hemi-molar amount of tafamidis, fumaric acid, and a suitable solvent. For instance, tafamidis (about 1 mmol), fumaric acid (about 2 mmol), and a suitable solvent (e.g., about 18 mL of acetone) may be combined in a suitable container (e.g., a 25-mL glass vial) to form a suspension. The suspension may be stirred at room temperature for a suitable period (e.g., about 5 days), and then collected by filtration. After separation, the obtained solid may be dried under vacuum at room temperature for about one hour. A portion of the dried sample (e.g., about 270 mg) may be transferred into a separate suitable container (e.g., a 25-mL glass vial) along with a suitable amount of water (e.g., about 5 mL) followed by stirring at room temperature for about one hour. An additional portion of water (e.g., about 4 mL) may be added and the suspension may be stirred for a suitable period (e.g., 18-24 hours) to obtain a solid form comprising tafamidis and fumaric acid, which may be separated by filtration and dried under vacuum for a period at a suitable temperature (e.g., about 25° C. to about 50° C.).

### Example 1. Tafamidis Meglumine Capsules

Table 1 provides the compositional makeup of a capsule composition (Ex. 1) comprising tafamidis meglumine and at least one pharmaceutically acceptable excipient.

TABLE 1
Compositional makeup of Example 1

| Ingredients(s) | mg/capsule |
|---|---|
| Tafamidis meglumine | 20.00 |
| Lactose monohydrate | 108.00 |
| Maize starch | 5.00 |
| Croscarmellose sodium | 5.00 |
| Syloid 244 FP | 3.00 |
| Sodium lauryl sulfate | 1.00 |
| Magnesium stearate | 1.00 |
| Capsule fill weight | 200.00 |

### Manufacturing Process:

Accurately weighed tafamidis meglumine, sodium lauryl sulfate and syloid 244 FP (e.g., silica glident) were sifted using #30. Weighed lactose monohydrate, maize starch, croscarmellose sodium, were sifted using #20. Both #30 mesh and #20 mesh materials were blended for 15 min. The above blend was lubricated using #40 mesh sifted magnesium stearate for 5 min. The lubricated blend was filled into hard gelatin capsules with average fill weight of 200.00 mg. The dissolution testing was performed for capsules using USP II (paddle) apparatus, 900 mL of pH 6.8 phosphate buffer at 75 rpm.

### Example 2. Tafamidis Meglumine Capsules

Table 2 provides the compositional makeup of a capsule composition (Ex. 2) comprising tafamidis meglumine and at least one pharmaceutically acceptable excipient.

TABLE 2
Compositional makeup of Example 2

| Ingredients(s) | mg/capsule |
|---|---|
| Tafamidis meglumine | 20.00 |
| Povidone K30 | 10.00 |
| Sodium lauryl sulfate | 4.00 |
| Polysorbate 80 | 2.50 |
| Purified water | qs |
| Lactose monohydrate | 369.50 |
| Crospovidone | 15.00 |
| Colloidal silicon dioxide | 4.00 |
| Capsule fill weight | 450.00 |

### Manufacturing Process:

Accurately weighed tafamidis meglumine, sodium lauryl sulfate, polysorbate 80, povidone K30 were added into purified water and mixed well. Weighed lactose monohydrate and crospovidone were sifted using #20. Mesh #20 sifted materials were granulated (top spray granulation) using tafamidis meglumine dispersion. The above blend was mixed with #40 mesh sifted colloidal silicon dioxide for 5 min. The blend was filled into hard gelatin capsules with average fill weight of 450.00 mg. The dissolution testing was performed for capsules using USP II (paddle) apparatus, 900 mL of pH 6.8 phosphate buffer at 75 rpm.

### Example 3. Tafamidis Meglumine Capsules

Table 3 provides the compositional makeup of a capsule composition (Ex. 3) comprising tafamidis meglumine and at least one pharmaceutically acceptable excipient.

TABLE 3
Compositional makeup of Example 3

| | Example 3 |
|---|---|
| Ingredients(s) | mg/capsule |
| Tafamidis meglumine | 20.00 |
| Povidone K30 | 20.00 |
| Polacrillin potassium | 10.00 |
| Mannitol SD 200 | 147.40 |
| Colloidal silicon dioxide | 2.00 |
| Magnesium Stearate | 0.60 |
| Capsule fill weight | 200.00 |

### Manufacturing Process:

Accurately weighed tafamidis meglumine, polacrillin potassium, mannitol SD 200, povidone K30 were added into purified water and mixed for 30 min to form complex with povidone K30. The resulting dispersion were dried in tray dryer and milled. The above milled materials were mixed with #40 mesh sifted colloidal silicon dioxide and magnesium stearate for 5 min. The blend was filled into hard gelatin capsules with average fill weight of 200.00 mg. The dissolution testing was performed for capsules using USP II (paddle) apparatus, 900 mL of pH 6.8 phosphate buffer at 75 rpm.

### Example 4. Tafamidis Meglumine Capsules

Table 4 provides the compositional makeup of a capsule composition (Ex. 4) comprising tafamidis meglumine and at least one pharmaceutically acceptable excipient.

TABLE 4
Compositional makeup of Example 4

| | Example 4 |
|---|---|
| Ingredients(s) | mg/capsule |
| Tafamidis meglumine | 20.00 |
| Poloxamer 188 | 20.00 |
| Croscarmellose sodium | 10.00 |
| Mannitol SD 200 | 147.40 |
| Colloidal silicon dioxide | 2.00 |
| Magnesium Stearate | 0.60 |
| Capsule fill weight | 200.00 |

### Manufacturing Process:

Accurately weighed tafamidis meglumine, poloxamer P188, croscarmellose sodium, mannitol SD 200 were added into purified water and mixed for 30 min. The resulting dispersion were dried in tray dryer and milled. The above milled materials were mixed with #40 mesh sifted colloidal silicon dioxide and magnesium stearate for 5 min. The blend was filled into hard gelatin capsules with average fill weight of 200.00 mg.

Table 5 presents dissolution test results for the compositions of Examples 1-2. The dissolution testing was performed for capsules using USP II (paddle) apparatus, 900 mL of pH 6.8 phosphate buffer at 75 rpm.

TABLE 5
Dissolution Data for Examples 1-2

| | Dissolution at 50 mM Sodium phosphate | | | | | | | |
|---|---|---|---|---|---|---|---|---|
| | buffer pH 6.8, 900 mL, 75 rpm, USP II | | | | | | | |
| | (Paddle) Q point at 30 min | | | | | | | |
| | % drug release | | | | | | | |
| | 5 | 10 | 15 | 20 | 30 | 45 | 60 | Obser- |
| Example(s) | min | min | min | min | min | min | min | vation |
| Example 1 | 2 | 3 | 6 | 5 | 8 | 9 | 12 | A rigid |
| Example 2 | — | 24 | 20 | 24 | 32 | 34 | 44 | gel |
| | | | | | | | | formed |

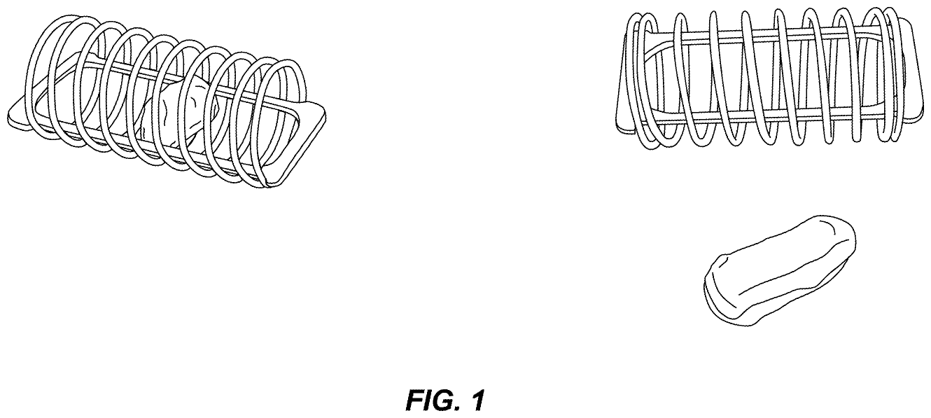

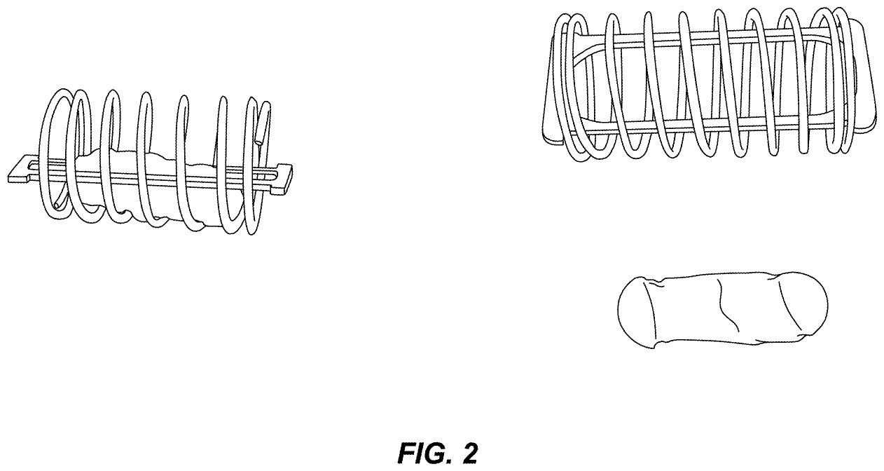

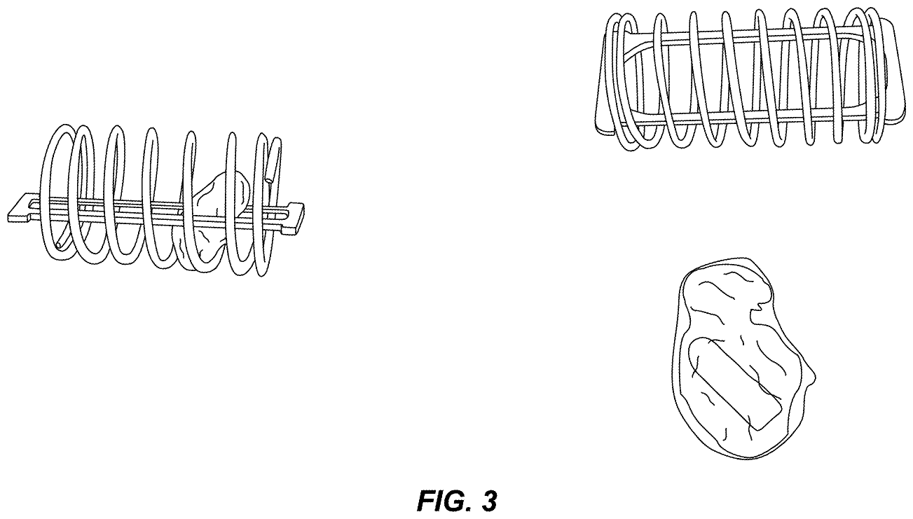

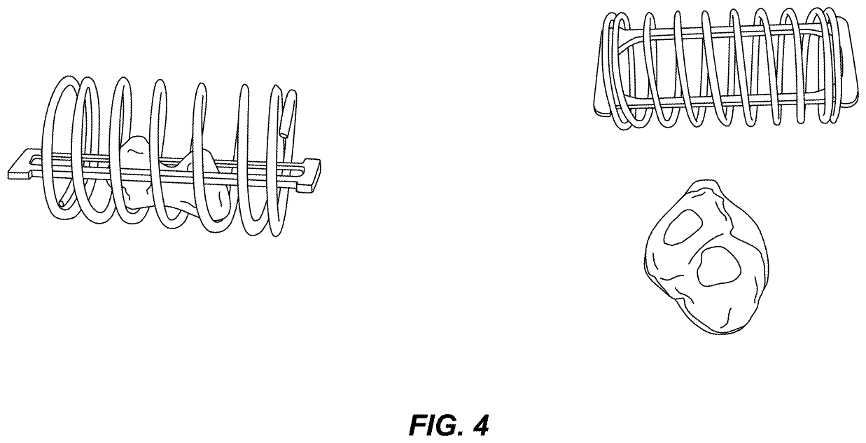

The Table 5 data shows that a rigid gel formed for the capsule compositions of Examples 1-2. FIGS. 1-4 depict the observed capsule gels formed for Examples 1-4, respectively. Hard gelatin capsule formulations containing granules with the identified excipients, formed a rigid gel structure upon contact with aqueous vehicles, i.e., pH 6.8 phosphate buffer thereby affecting the dissolution rate or drug release rate from the solid dosage.

### Examples 5-7. Tafamidis Meglumine Capsules (Organic Acidifier Granules)

Table 6 provides the compositional makeup of capsule compositions (Exs. 5-7) comprising tafamidis meglumine and at least one pharmaceutically acceptable excipient.

TABLE 6
Compositional makeup of Examples 5-7 (granules)

| | Example 5 | Example 6 | Example 7 |
|---|---|---|---|
| Ingredients(s) | mg/capsule | | |
| Tafamidis meglumine | 20.00 | 20.00 | 20.00 |
| Mannitol SD 200 | 347.00 | — | — |
| Lactose monohydrate | — | 422.50 | — |
| Microcrystalline cellulose | — | — | 382.25 |
| Povidone K30 | — | 15.00 | 9.00 |
| Croscarmellose sodium | 20.00 | 25.00 | 22.50 |
| Sodium lauryl sulfate | 2.00 | 5.00 | 4.50 |
| Citric acid anhydrous | 5.00 | 5.00 | 5.00 |
| Silicon dioxide | 4.00 | 5.00 | 4.50 |
| Magnesium Stearate | 4.00 | 2.50 | 2.25 |
| Capsule fill weight | 402.00 | 500.00 | 450.00 |

### Manufacturing Process for Example 5:

Accurately weighed Tafamidis meglumine, mannitol SD 200, croscarmellose sodium, sodium lauryl sulfate was sifted using #30. Mesh #30 sifted materials were granulated in High shear mixer granulator using citric acid anhydrous solution. The wet mass was dried and milled, the dried blend was mixed with #40 mesh sifted colloidal silicon dioxide and magnesium stearate for 5 min. The blend was filled into hard gelatin capsules with average fill weight of 402.00 mg. The dissolution testing was performed for capsules using USP II (paddle) apparatus, 900 mL of pH 6.8 phosphate buffer at 75 rpm.

### Manufacturing Process for Example 6:

Accurately weighed Tafamidis meglumine, lactose monohydrate, povidone K30, croscarmellose sodium, sodium lauryl sulfate was sifted using #30. Mesh #30 sifted materials were granulated in High shear mixer granulator using citric acid anhydrous solution. The wet mass was dried and milled, the dried blend was mixed with #40 mesh sifted colloidal silicon dioxide and magnesium stearate for 5 min. The blend was filled into hard gelatin capsules with average fill weight of 500.00 mg. The dissolution testing was performed for capsules using USP II (paddle) apparatus, 900 mL of pH 6.8 phosphate buffer at 75 rpm.

### Manufacturing Process for Example 7:

Accurately weighed Tafamidis meglumine, microcrystalline cellulose, povidone K30, croscarmellose sodium, sodium lauryl sulfate was sifted using #30. Mesh #30 sifted materials were granulated in High shear mixer granulator using citric acid anhydrous solution. The wet mass was dried and milled, the dried blend was mixed with #40 mesh sifted colloidal silicon dioxide and magnesium stearate for 5 min. The blend was filled into hard gelatin capsules with average fill weight of 450.00 mg.

Table 7 presents dissolution test results for the compositions of Examples 5-7. The dissolution testing was performed for capsules using USP II (paddle) apparatus, 900 mL of pH 6.8 phosphate buffer at 75 rpm.

TABLE 7
Dissolution Data for Examples 5-7

| | Dissolution at 50 mM Sodium phosphate | | | | | | | |
|---|---|---|---|---|---|---|---|---|
| | buffer pH 6.8, 900 mL, 75 rpm, USP II | | | | | | | |
| | (Paddle), Q point at 30 min | | | | | | | |
| | % drug release | | | | | | | |
| | 5 | 10 | 15 | 20 | 30 | 45 | 60 | Obser- |
| Example(s) | min | min | min | min | min | min | min | vation |
| Example 5 | 64 | 79 | 84 | 88 | 90 | 93 | 94 | No gel |
| Example 6 | 65 | 84 | 90 | 92 | 95 | 96 | 97 | forma- |
| Example 7 | 34 | 70 | 79 | 85 | 90 | 93 | 95 | tions |
| | | | | | | | | were |
| | | | | | | | | ob- |
| | | | | | | | | served |

Based on the Table 7 data, it may be seen that, e.g., Example 6 shows the method of manufacturing a solid composition with an acidifier in granules filled into hard gelatin capsules overcomes the gel formation in dissolution preferably in tafamidis meglumine capsules 20 mg official media (OGD) of 50 mM Sodium phosphate buffer pH 6.8, 900 mL, 75 rpm, USP II (Paddle), Q point at 30 min.

Examples 5-7 demonstrate a solid pharmaceutical formulations having various composed granules with acidifiers filled into hard gelatin capsules makes the tafamidis meglumine granules to disperse easily or within 5 to 7 minutes without the formation of a gel or gel structured mucilage formation upon contact with water or any buffer solution preferably in tafamidis meglumine capsules 20 mg official media (OGD) of 50 mM Sodium phosphate buffer pH 6.8, 900 mL, 75 rpm, USP II (Paddle), Q point at 30 min.

### Example 8. Tafamidis Meglumine Capsules (Organic Acidifier Granules)

Table 6 provides the compositional makeup of a capsule composition (Ex. 8) comprising tafamidis and at least one pharmaceutically acceptable excipient.

TABLE 8
Compositional makeup of Example 8

| | Example 8 |
|---|---|
| Ingredients(s) | mg/capsules |
| Tafamidis | 61.00 |
| Lactose monohydrate | 294.00 |
| Povidone K30 | 10.00 |
| Croscarmellose sodium | 20.00 |
| Sodium lauryl sulfate | 4.00 |
| Citric acid anhydrous | 5.00 |
| Silicon dioxide | 4.00 |
| Magnesium Stearate | 2.00 |
| Capsule fill weight | 400.00 |

### Manufacturing Process:

Accurately weighed tafamidis, lactose monohydrate, povidone K30, croscarmellose sodium, sodium lauryl sulfate was sifted using #30. Mesh #30 sifted materials were granulated in High shear mixer granulator using citric acid anhydrous solution. The wet mass was dried and milled, the dried blend was mixed with #40 mesh sifted colloidal silicon dioxide and magnesium stearate for 5 min. The blend was filled into hard gelatin capsules with average fill weight of 400.00 mg.

Table 9 presents dissolution test results for the composition of Example 8. The dissolution testing was performed for capsules using USP II (paddle) apparatus, 900 mL of pH 6.8 phosphate buffer at 75 rpm.

TABLE 9
Dissolution data for Example 8

| | Dissolution at 50 mM Sodium phosphate | | | | | | | |
|---|---|---|---|---|---|---|---|---|
| | buffer pH 6.8 + 1% Tween 80, 900 mL, | | | | | | | |
| | 75 rpm, USP II (Paddle), Q point at 30 min | | | | | | | |
| | % drug release | | | | | | | |
| | 5 | 10 | 15 | 20 | 30 | 45 | 60 | Obser- |
| Example | min | min | min | min | min | min | min | vation |
| Example 8 | 64 | 79 | 84 | 88 | 90 | 93 | 94 | No gel |
| | | | | | | | | forma- |
| | | | | | | | | tions |
| | | | | | | | | were ob- |
| | | | | | | | | served |

Based on the Table 9 data, it may be seen that the Example 8 composition shows the method of manufacturing a solid composition with acidifying agent in granules filled into hard gelatin capsules overcomes the gel formation in dissolution preferably in tafamidis capsules 61 mg official media (OGD) of 50 mM Sodium phosphate buffer pH 6.8+1% Tween 80, 900 mL, 75 rpm, USP II (Paddle), Q point at 30 min.

Example 8 demonstrates a solid pharmaceutical formulations comprising granules with acidifiers filled into hard gelatin capsules makes the tafamidis granules to disperse easily (e.g., within 5 to 7 minutes) without the formation of a gel or gel structured mucilage formation upon contact with water or any buffer solution preferably in tafamidis capsules 61 mg official media (OGD) of 50 mM Sodium phosphate buffer pH 6.8+1% Tween 80, 900 mL, 75 rpm, USP II (Paddle), Q point at 30 min.

### Example 9. Tafamidis Meglumine Capsules

Table 10 provides the compositional makeup of a capsule composition (Ex. 9) comprising tafamidis meglumine and at least one pharmaceutically acceptable excipient.

TABLE 10
Compositional makeup of Example 9

| Ingredients(s) | mg/capsule |
|---|---|
| Tafamidis meglumine | 20.00 |
| Lactose monohydrate | 108.00 |
| Maize starch | 5.00 |
| Croscarmellose sodium | 5.00 |
| Syloid 244 FP | 3.00 |
| Sodium lauryl sulfate | 1.00 |
| Magnesium stearate | 1.00 |
| Capsule fill weight | 200.00 |

### Example 10. Tafamidis Meglumine Capsules

Table 11 provides the compositional makeup of a capsule composition (Ex. 10) comprising tafamidis meglumine and at least one pharmaceutically acceptable excipient.

TABLE 11
Compositional makeup of Example 10

| Ingredients(s) | mg/capsule |
|---|---|
| Tafamidis meglumine | 20.00 |
| Hydroxypropyl-cyclodextrin | 20.00 |
| Sodium lauryl sulfate | 0.20 |
| Purified water | qs |
| Lactose monohydrate | 74.04 |
| Crospovidone | 4.80 |
| Colloidal silicon dioxide | 0.96 |
| Capsule fill weight | 120.00 |

Table 12 presents dissolution test results for the compositions of Examples 9-10.

TABLE 12
Dissolution Data for Examples 9-10

| | Dissolution at 50 mM | | | | | | | |
|---|---|---|---|---|---|---|---|---|
| | Sodium phosphate buffer pH 6.8, | | | | | | | |
| | 900 mL, 75 rpm, USP II (Paddle) | | | | | | | |
| | % drug release | | | | | | | |
| | 5 | 10 | 15 | 20 | 30 | 45 | 60 | Obser- |
| Example(s) | min | min | min | min | min | min | min | vation |
| Example 9 | 2 | 3 | 6 | 5 | 8 | 9 | 12 | Rigid |
| Example 10 | — | 24 | 20 | 24 | 32 | 34 | 44 | gel |
| | | | | | | | | formed |

### Examples 11-12. Tafamidis Meglumine Capsules (Spray-Dried)

Table 13 provides the compositional makeup of a capsule compositions (Exs. 11-12) comprising tafamidis meglumine and at least one pharmaceutically acceptable excipient.

TABLE 13
Compositional makeup of Examples
11-12 (spray-dried)

| | Example 11 | Example 12 |
|---|---|---|
| Ingredients(s) | mg/capsule | |
| Tafamidis meglumine | 20.00 | 20.00 |
| Hydroxypropyl β-cyclodextrin | 20.00 | 100.00 |
| Purified water | qs | qs |
| Capsule fill weight | 40.00 | 120.00 |

Table 14 presents dissolution test results for the compositions of Examples 11-12.

TABLE 14
Dissolution data for Examples 11-12

| | Dissolution at 50 mM | | | | | | | |
|---|---|---|---|---|---|---|---|---|
| | Sodium phosphate buffer pH 6.8, | | | | | | | |
| | 900 mL, 75 rpm, USP II (Paddle) | | | | | | | |
| | % drug release | | | | | | | |
| | 5 | 10 | 15 | 20 | 30 | 45 | 60 | Obser- |
| Example(s) | min | min | min | min | min | min | min | vation |
| Example 11 | 5 | 11 | 16 | 18 | 23 | 26 | 29 | Rigid |
| | | | | | | | | gel |
| | | | | | | | | formed |
| Example 12 | — | 94 | 97 | 98 | 97 | 97 | 97 | No gel |
| | | | | | | | | forma- |
| | | | | | | | | tion ob- |
| | | | | | | | | served |

### Examples 13-14. Tafamidis Meglumine Capsules (Spray-Dried)

Table 11 provides the compositional makeup of capsule compositions (Exs. 13-14) comprising tafamidis meglumine and at least one pharmaceutically acceptable excipient.

TABLE 15
Compositional makeup of Examples 13-14 (spray-dried)

| | Example 13 | Example 14 |
|---|---|---|
| Ingredients(s) | mg/capsule | |
| Tafamidis meglumine | 20.00 | 20.00 |
| β-cyclodextrin | 100.00 | 100.00 |
| Lactose monohydrate | 68.00 | 68.00 |
| Crospovidone | 10.00 | 10.00 |
| Purified water | qs | qs |
| Capsule fill weight | 198.00 | 198.00 |
| | Tafamidis | Tafamidis |
| | meglumine + | meglumine + |
| | β-cyclodextrin | β-cyclodextrin |
| | dissolved in | dispersed in |
| | required | less |
| | quantity | quantity |
| | of water | of water |

Table 16 presents dissolution test results for the compositions of Examples 13-14.

TABLE 16
Dissolution data for Examples 13-14

| | Dissolution at 50 mM | | | | | | | |
|---|---|---|---|---|---|---|---|---|
| | Sodium phosphate buffer pH 6.8, | | | | | | | |
| | 900 mL, 75 rpm, USP II (Paddle) | | | | | | | |
| | % drug release | | | | | | | |
| | 5 | 10 | 15 | 20 | 30 | 45 | 60 | Obser- |
| Example(s) | min | min | min | min | min | min | min | vation |
| Example 13 | 41 | 80 | 93 | 95 | 96 | 96 | 95 | No gel |
| Example 14 | 65 | 92 | 94 | 94 | 94 | 95 | 95 | forma- |
| | | | | | | | | tion ob- |
| | | | | | | | | served |

### Example 15. Tafamidis Capsules (Spray-Dried)

Table 17 provides the compositional makeup of a capsule composition (Ex. 15) comprising tafamidis and at least one pharmaceutically acceptable excipient.

TABLE 17
Compositional makeup of Example 15 (spray-dried)

| | Example 15 |
|---|---|
| Ingredients(s) | mg/capsule |
| Tafamidis | 61.00 |
| β-cyclodextrin | 305.00 |
| Lactose monohydrate | 61.50 |
| Crospovidone | 22.50 |
| Purified water | qs |
| Capsule fill weight | 450.00 |
| | Tafamidis + β-cyclodextrin |
| | dispersed in required quantity |
| | of water |

Table 18 presents dissolution test results for the composition of Example 15.

TABLE 18
Dissolution Data for Example 15

| | Dissolution at 50 mM | | | | | | | |
|---|---|---|---|---|---|---|---|---|
| | Sodium phosphate buffer pH 6.8, | | | | | | | |
| | 900 mL, 75 rpm, USP II (Paddle) | | | | | | | |
| | % drug release | | | | | | | |
| | 5 | 10 | 15 | 20 | 30 | 45 | 60 | Obser- |
| Example(s) | min | min | min | min | min | min | min | vation |
| Example 15 | 38 | 82 | 97 | 98 | 98 | 98 | 99 | No gel |
| | | | | | | | | forma- |
| | | | | | | | | tion ob- |
| | | | | | | | | served |

Table 19 provides a qualitative composition of tafamidis Tablets (61 mg) and tafamidis meglumine Tablets (20 mg).

TABLE 19
Qualitative Tablet Compositions

| Tafamidis | Tafamidis meglumine | Function |
|---|---|---|
| Tablets 61 mg | Tablets 20 mg | category |
| Tafamidis | Tafamidis meglumine | API |
| Lactose monohydrate | Lactose monohydrate | Diluent |
| Microcrystalline cellulose | Microcrystalline cellulose | Diluent |
| Croscarmellose sodium | Croscarmellose sodium | Disintegrant |
| Sodium Lauryl Sulfate | Sodium Lauryl Sulfate | Surfactant |
| Citric acid anhydrous | Citric acid anhydrous | Acidifier |
| Povidone | Povidone | Binder |
| Colloidal silicon dioxide | Colloidal silicon dioxide | Glidant |
| Magnesium stearate | Magnesium stearate | Lubricant |

### Example 16. Tafamidis Meglumine Tablets (with Lactose Monohydrate and Microcrystalline Cellulose)

Tafamidis meglumine Tablets 20 mg prototype composition with lactose monohydrate 200 mesh and microcrystalline cellulose (Vivapur PH 101) with povidone (Povidone K30) as binder by wet granulation process. Table 20 provides the compositional makeup of the Example 15 tablet composition.

TABLE 20
Compositional makeup of Example 16

| | |
|---|---|
| | Example 16 |
| Ingredients | mg/tablet |

Intra-granular portion

| | |
|---|---|
| Tafamidis meglumine | 20.00 |
| Lactose monohydrate 200 mesh | 65.30 |
| Microcrystalline cellulose (Vivapur pH 101) | 40.00 |
| Croscarmellose sodium (Vivasol) | 3.50 |
| Sodium lauryl sulfate (Stepanol WA-100) | 1.40 |
| Granulating fluid | |
| Purified water | qs |
| Povidone (Kollidon K30) | 3.50 |

Extra-granular portion

| | |
|---|---|
| Croscarmellose sodium (Vivasol) | 3.50 |
| Colloidal silicon dioxide (Aerosil 200) | 1.40 |
| Magnesium stearate | 1.40 |
| Total Tablet weight | 140.00 |

Executed batch size-1000 tablets

### Brief Manufacturing Procedure:

Step 1: All the raw materials were dispensed as per batch size quantity.

Step 2: The intragranular portion (tafamidis meglumine, lactose monohydrate, microcrystalline cellulose, croscarmellose sodium, and sodium lauryl sulfate) were sifted using #25 mesh and mixed for 10 minutes.

Step 3: Povidone K30 was dissolved in Purified water.

Step 4: Step 2 obtained materials were granulated using povidone binder solution and extra purified water were added until a suitable wet mass were obtained.

Step 5: Wet mass were dried in tray dryer at 60±5° C. until to reach the Loss on drying (LOD) NMT 2.0% w/w.

Step 6: Dried granules were sifted using #25 mesh and retains were milled if required.

Step 7: Croscarmellose sodium (extragranular) was sifted through #25 mesh and blended with step 6 obtained materials for 10 minutes.

Step 8: Step 7 obtained materials were blended with #40 mesh passed colloidal silicon dioxide and Magnesium stearate for 5 minutes.

Step 9: The blend was compressed using suitable tooling with the average tablet weight 140.00 mg.

Table 21 presents dissolution test results for the composition of Example 16 using different dissolution media.

TABLE 21
Dissolution Data for Example 16

| Dissolution condition: 900 mL, 75 rpm, USP II (Paddle) | | | |
|---|---|---|---|
| Official Media | | | |
| pH 6.8 | | | |
| Phosphate | 0.1N | pH 4.5 Acetate | |
| buffer | HCl | Buffer | Purified Water |

| | |
|---|---|
| Example 16 | Tablets not disintegrated/dispersed and found to be intact |
| | till 60 min. Tablet found to be slightly swelled due to |
| | gelation (rigid gel) with the contact of media. |

Based the Table 21 data of tablets in different dissolution media, it was observed that tablets containing tafamidis meglumine forms gel when contacts with water or other dissolution media.

### Example 17. Tafamidis Meglumine Tablets ((Organic Acidifier)

To prevent the gel formation of tablets containing tafamidis meglumine 20 mg, it was decided to acidify the blend using acidifier which may help to disperse the tablets in aqueous buffer media. The prototype composition is presented in below Table 22.

TABLE 22
Prototype composition

| | |
|---|---|
| Example 17A | Example 17B |

| | |
|---|---|
| Ingredient | mg/tablet |

Intra-granular portion

| | | |
|---|---|---|
| Tafamidis meglumine | 20.00 | 20.00 |
| Lactose monohydrate 200 mesh | 61.70 | 104.00 |
| Microcrystalline Cellulose (Vivapur PH 101) | 40.00 | — |
| Croscarmellose sodium (Vivasol) | 2.80 | 7.00 |
| Sodium lauryl sulfate (Stepanol WA-100) | 1.40 | 1.40 |
| Povidone (Kollidon K30) | 3.50 | 3.50 |

Granulating fluid

| | | |
|---|---|---|
| Purified water | qs | qs |
| Citric acid anhydrous | 5.00 | 2.00 |
| Extra-granular portion | | |
| Croscarmellose sodium (Vivasol) | 2.80 | — |
| Colloidal silicon dioxide (Aerosil 200) | 1.40 | 1.40 |
| Magnesium stearate | 1.40 | 1.40 |
| Total Tablet weight | 140.00 | 140.00 |

Executed batch size-1000 tablets

### Brief Manufacturing Procedure:

Step 1: All the raw materials were dispensed as per batch size quantity.

Step 2: Intragranular portion (tafamidis meglumine, lactose monohydrate, microcrystalline cellulose, croscarmellose sodium, sodium lauryl sulfate, povidone) were sifted using #25 mesh and mixed for 10 minutes.

Step 3: Citric acid anhydrous was dissolved in Purified water to provide granulating fluid.

Step 4: Step 2 obtained materials were granulated using citric acid solution and extra purified water were added until a suitable wet mass were obtained.

Step 5: Wet mass were dried in tray dryer at 60±5° C. until to reach the Loss on drying (LOD) NMT 2.0% w/w.

Step 6: Dried granules were sifted using #25 mesh and retains were milled in fitz mill fitted with 0033 screen if required.

Step 7: Croscarmellose sodium (extragranular) was sifted through #25 mesh and blended with step 6 obtained materials for 10 minutes.

Step 8: Step 7 obtained materials were blended with #40 mesh passed colloidal silicon dioxide and Magnesium stearate for 5 minutes.

Step 9: The blend was compressed using suitable tooling with the average tablet weight 140.00 mg.

The compressed tablets were subjected to physical observation in different dissolution media. The observations are presented below.

TABLE 23a
Dissolution data (pH 6.8 and 0.1N HCl) for Examples 17A-17B

| | Dissolution condition: 900 mL, 75 rpm, USP II (Paddle) | | | |
|---|---|---|---|---|
| | pH 6.8 Phosphate buffer | | 0.1N HCl | |
| | | Tablet | | Tablet |
| | Gel | Disintegration/ | Gel | Disintegration/ |
| | formation | Dispersion | formation | Dispersion |
| | Yes/No | time | Yes/No | time |
| Example | No gelation | Within | No gelation | within |
| 17B | observed | 7 min | observed | 10 min |
| Example | No gelation | Within | No gelation | within |
| 17A | observed | 7 min | observed | 10 min |

TABLE 23b
Dissolution data (pH 4.5 and purified water) for Examples 17A-17B

| | Dissolution condition: 900 mL, 75 rpm, USP II (Paddle) | | | |
|---|---|---|---|---|
| | pH 4.5 Acetate buffer | | Purified water | |
| | | Tablet | | Tablet |
| | Gel | Disintegration/ | Gel | Disintegration/ |
| | formation | Dispersion | formation | Dispersion |
| | Yes/No | time | Yes/No | time |
| Example | No gelation | Within | No gelation | Within |
| 17B | observed | 6 min | observed | 7 minutes |
| Example | No gelation | Within | No gelation | Within |
| 17A | observed | 6 min | observed | 7 minutes |

### Selection of Dissolution Condition

Based on approval history (USFDA) of tafamidis meglumine soft gelatin capsules 20 mg, the recommended dissolution condition for drug release media is 50 mM Sodium Phosphate buffer pH 6.8, 900 mL, 75 rpm, USP II (Paddle). USP 33<711>. Accordingly, the same dissolution conditions were chosen as drug release media for tafamidis meglumine tablets 20 mg, i.e., 50 mM Sodium Phosphate buffer pH 6.8, 900 mL, 75 rpm, USP II (Paddle). All trial batches were evaluated in this media for drug release.

In addition to this above-mentioned release media, dissolution was evaluated in purified water to find the drug release rate. Due to poor solubility/insoluble nature of tafamidis meglumine, dissolution was not performed in media like 0.1 N HCl and pH 4.5 acetate buffer.

TABLE 24
Dissolution data (official media and
purified water) for Example 17A

| | Batch no. | | | | | | | |
|---|---|---|---|---|---|---|---|---|
| | 5 | 10 | 15 | 20 | 30 | 45 | 60 | Inf |
| | Official Media: 50 mM Sodium Phosphate | | | | | | | |
| | buffer pH 6.8, 900 mL, 75 rpm, USP II (Paddle) | | | | | | | |
| Example 17A | 49 | 62 | 71 | 77 | 83 | 90 | 93 | 96 |
| | Purified water, 900 mL, 75 rpm, USP II (Paddle) | | | | | | | |
| Example 17A | 15 | 18 | 17 | 20 | 22 | 22 | 22 | 21 |

Based on the Table 24 data, it was observed that in the presence of citric acid anhydrous in tablets prevents the gelation phenomena of tablets containing tafamidis meglumine when tablets contacted water or other dissolution media. A possible reason for disintegration/dispersion of tablets into particles may be due to acidifying the blend helps tablets to disperse in buffer medias. In the dissolution, drug release found to be more than 80% w/w at 30 min time point in pH 6.8 phosphate buffer and Purified water. Hence, it was decided to execute the similar experimental trials using various acidifying excipients.

### Examples 18-21. Tafamidis Meglumine Tablets (Organic Acidifiers)

In view of the foregoing results, different organic acidifiers (e.g., tartaric acid, malic acid, lactic acid, and fumaric acid) were evaluated to find the effect of acidifier on dispersion of tablets. The compositional makeup of Exs. 18-19 comprising different acidifiers are presented in Table 25.

TABLE 25
Compositional makeup of Examples 18-21

| | Example | Example | Example | Example |
|---|---|---|---|---|
| Example No. | 18 | 19 | 20 | 21 |

| | |
|---|---|
| Ingredient(s) | mg/tablet |

Intra-granular portion

| | | | | |
|---|---|---|---|---|
| Tafamidis meglumine | 20.00 | 20.00 | 20.00 | 20.00 |
| Lactose monohydrate 200 mesh | 61.70 | 61.70 | 64.70 | 61.70 |
| Microcrystalline Cellulose | 40.00 | 40.00 | 40.00 | 40.00 |
| (Vivapur PH 101) | | | | |
| Croscarmellose sodium (Vivasol) | 2.80 | 2.80 | 2.80 | 2.80 |
| Sodium lauryl sulfate (Stepanol | 1.40 | 1.40 | 1.40 | 1.40 |
| WA-100) | | | | |
| Povidone (Kollidon K30) | 3.50 | 3.50 | 3.50 | 3.50 |

Granulating fluid

| | | | | |
|---|---|---|---|---|
| Purified water | qs | qs | qs | — |
| Absolute alcohol | — | — | — | qs |
| Tartaric acid | 5.00 | — | — | — |
| Malic acid | — | 5.00 | — | — |
| Lactic acid | — | — | 2.00 | — |
| Fumaric acid | — | — | — | 5.00 |

Extra-granular portion

| | | | | |
|---|---|---|---|---|
| Croscarmellose sodium (Vivasol) | 2.80 | 2.80 | 2.80 | 2.80 |
| Colloidal silicon dioxide (Aerosil | 1.40 | 1.40 | 1.40 | 1.40 |
| 200) | | | | |
| Magnesium stearate | 1.40 | 1.40 | 1.40 | 1.40 |
| Total Tablet weight | 140.00 | 140.00 | 140.00 | 140.00 |

Executed trial batch size-Each batch 1000 tablets

### Brief Manufacturing Procedure:

Step 1: All the raw materials were dispensed as per batch size quantity.

Step 2: Intragranular portion (tafamidis meglumine, lactose monohydrate, microcrystalline cellulose, croscarmellose sodium, sodium lauryl sulfate, povidone) were sifted using #25 mesh and mixed for 10 minutes.

Step 3: The respective batch acidifier was dissolved in purified water or alcohol providing granulating fluid.

Step 4: Step 2 obtained materials were granulated using respective batch acidifier containing solution and extra granulating fluid were added until a suitable wet mass were obtained.

Step 5: Wet mass were dried in tray dryer at 60±5° C. until to reach the Loss on drying (LOD) NMT 2.0% w/w.

Step 6: Dried granules were sifted using #25 mesh and retains were milled if required.

Step 7: Croscarmellose sodium (extragranular) was sifted through #25 mesh and blended with step 6 obtained materials for 10 minutes.

Step 8: Step 7 obtained materials were blended with #40 mesh passed colloidal silicon dioxide and magnesium stearate for 5 minutes.

Step 9: The blend was compressed using suitable tooling with the average tablet weight 140.00 mg.

TABLE 26a
Dissolution data (pH 6.8 and 0.1N HCl) for Examples 18-21

| | Dissolution condition: 900 mL, 75 rpm, USP II (Paddle) | | | |
|---|---|---|---|---|
| | pH 6.8 Phosphate buffer | | 0.1N HCl | |
| | | Tablet | | Tablet |
| | Gel | Disintegration/ | Gel | Disintegration/ |
| | formation | Dispersion | formation | Dispersion |
| | Yes/No | time | Yes/No | time |
| Example 18 | No gelation | Within 6min | No gelation | within 3 min |
| | observed | | observed | |
| Example 19 | No gelation | Within 4min | No gelation | within 3 min |
| | observed | | observed | |
| Example 20 | No gelation | Within 4min | No gelation | within 4 min |
| | observed | | observed | |
| Example 21 | No gelation | Within 6min | No gelation | within 3 min |
| | observed | | observed | |

TABLE 26b
Dissolution data (pH 4.5 and purified water) for Examples 18-21

| | Dissolution condition: 900 mL, 75 rpm, USP II (Paddle) | | | |
|---|---|---|---|---|
| | pH 4.5 Acetate buffer | | Purified water | |
| | | Tablet | | Tablet |
| | Gel | Disintegration/ | Gel | Disintegration/ |
| | formation | Dispersion | formation | Dispersion |
| | Yes/No | time | Yes/No | time |
| Example 18 | No gelation | Within 5 min | No gelation | within 5 min |
| | observed | | observed | |
| Example 19 | No gelation | Within 2 min | No gelation | within 2 min |
| | observed | | observed | |
| Example 20 | No gelation | Within 4 min | No gelation | within 4 min |
| | observed | | observed | |
| Example 21 | No gelation | Within 5 min | No gelation | within 4 min |
| | observed | | observed | |

TABLE 26c
Dissolution data (official media) for Examples 18-21

| | Official Media: 50 mM | | | | | | | |
|---|---|---|---|---|---|---|---|---|
| | Sodium Phosphate buffer pH 6.8, | | | | | | | |
| | 900 mL, 75 rpm, USP II (Paddle) | | | | | | | |
| Exam. no. | 5 | 10 | 15 | 20 | 30 | 45 | 60 | Inf |
| Example 18 | 56 | 64 | 73 | 78 | 85 | 90 | 93 | 97 |
| Example 19 | 53 | 65 | 73 | 76 | 82 | 87 | 89 | 90 |
| Example 20 | 52 | 68 | 76 | 81 | 86 | 91 | 92 | 94 |
| Example 21 | 58 | 72 | 78 | 82 | 85 | 88 | 89 | 90 |

Based results of Tables 26a-26c, it was observed that in the presence of different organic acidifier like tartaric acid, malic acid, lactic acid and fumaric acid in tablets prevents the gelation phenomena of tablet containing tafamidis meglumine upon contact with water or other dissolution media. Hence, it was further conforming and clarifies the presence of an acidifier prevents the gelation phenomena of tablet containing tafamidis meglumine. A possible reason for disintegration/dispersion of tablets into particles may be due to acidifying the blend helps tables to disperse in buffer medias. Drug release found to be more than 80% w/w in 30 min time point in pH 6.8 phosphate buffer (official media).

### Examples 22-23. Tafamidis Meglumine Tablets (Inorganic Acidifiers)

Different inorganic acidifiers (e.g., hydrochloric acid and phosphoric acid) were evaluated. In order to break the gel formation of tablets containing Tafamidis meglumine 20 mg, it was decided to use different Inorganic acidifiers in the tablets were executed. The prototype composition is presented Table 27.

TABLE 27
Compositional makeup of Examples 22-23 (tablet)

| | |
|---|---|
| Example 22 | Example 23 |

| | |
|---|---|
| Ingredients | mg/tablet |

Intra-granular portion

| | | |
|---|---|---|
| Tafamidis meglumine | 20.00 | 20.00 |
| Lactose monohydrate 200 mesh | 61.70 | 61.70 |
| Microcrystalline Cellulose (Vivapur PH 101) | 40.00 | 40.00 |
| Croscarmellose sodium (Vivasol) | 2.80 | 2.80 |
| Sodium lauryl sulfate (Stepanol WA-100) | 1.40 | 1.40 |
| Povidone (Kollidon K30) | 3.50 | 3.50 |

Granulating fluid

| | | |
|---|---|---|
| Purified water | qs | qs |
| Hydrochloric acid | 2.50 | — |
| Phosphoric acid | — | 3.40 |

Extra-granular portion

| | | |
|---|---|---|
| Croscarmellose sodium (Vivasol) | 2.80 | 2.80 |
| Colloidal silicon dioxide (Aerosil 200) | 1.40 | 1.40 |
| Magnesium stearate | 1.40 | 1.40 |
| Total Tablet weight | 140.00 | 140.00 |

Executed trial batch size-Each batch 1000 tablets

Brief Manufacturing Procedure:

Step 1: All the raw materials were dispensed as per batch size quantity.

Step 2: Intragranular portion (tafamidis meglumine, lactose monohydrate, microcrystalline cellulose, croscarmellose sodium, sodium lauryl sulfate, and povidone) were sifted using #25 mesh and mixed for 10 minutes.

Step 3: The respective inorganic acidifier was dissolved in Purified water to provide granulating fluid.

Step 4: Step 2 obtained materials were granulated using respective batch acidifier containing solution and extra granulating fluid were added until a suitable wet mass were obtained.

Step 5: Wet mass were dried in tray dryer at 60±5° C. until to reach the Loss on drying (LOD) NMT 2.0% w/w.

Step 6: Dried granules were sifted using #25 mesh and retains were milled if required.

Step 7: Croscarmellose sodium (extragranular) was sifted through #25 mesh and blended with step 6 obtained materials for 10 minutes.

Step 8: Step 7 obtained materials were blended with #40 mesh passed colloidal silicon dioxide and Magnesium stearate for 5 minutes.

Step 9: The blend was compressed using suitable tooling with the average tablet weight 140.00 mg.

The compressed tablets were subjected to physical observation in different dissolution media. The observations were presented in Tables 28a-28c.

TABLE 28a
Dissolution Data (pH 6.8 and 0.1N HCl) for Examples 22-23

| | Dissolution condition: 900 mL, 75 rpm, USP II (Paddle) | | | |
|---|---|---|---|---|
| | pH 6.8 Phosphate buffer | | 0.1N HCl | |
| | | Tablet | | Tablet |
| | Gel | Disintegration/ | Gel | Disintegration/ |
| | formation | Dispersion | formation | Dispersion |
| | Yes/No | time | Yes/No | time |
| Example | No gelation | Within | No gelation | Within |
| 22 | observed | 7 min | observed | 7 min |
| Example | No gelation | Within | No gelation | Within |
| 23 | observed | 12 min | observed | 9 min |

TABLE 28b
Dissolution Data (pH 4.5 and purified water) for Examples 22-23

| | Dissolution condition: 900 mL, 75 rpm, USP II (Paddle) | | | |
|---|---|---|---|---|
| | pH 4.5 Acetate buffer | | Purified water | |
| | | Tablet | | Tablet |
| | Gel | Disintegration/ | Gel | Disintegration/ |
| | formation | Dispersion | formation | Dispersion |
| | Yes/No | time | Yes/No | time |
| Example 22 | No gelation | Within | No gelation | within |
| | observed | 5 min | observed | 5 min |
| Example 23 | No gelation | Within | No gelation | within |
| | observed | 7 min | observed | 12 min |

TABLE 28c
Dissolution data (official media) for Examples 22-23

| | Official Media: 50 mM | | | | | | | |
|---|---|---|---|---|---|---|---|---|
| | Sodium Phosphate buffer pH 6.8, | | | | | | | |
| | 900 mL, 75 rpm, USP II (Paddle) | | | | | | | |
| Batch no. | 5 | 10 | 15 | 20 | 30 | 45 | 60 | Inf |
| Example 22 | 42 | 59 | 69 | 76 | 83 | 90 | 94 | 96 |
| Example 23 | 43 | 58 | 68 | 69 | 75 | 80 | 84 | 88 |

Based on results shown in Tables 28a-28c, it was observed that in the presence of different inorganic acidifier (e.g., hydrochloric acid and phosphoric acid) in tablets would overcome the gelation phenomena of tablet containing tafamidis meglumine upon contact with water or other dissolution media. Hence, these results confirm that the presence of any inorganic acidifier would prevent the gelation of tablet containing tafamidis meglumine. Drug release found to be more than 75% w/w in 30 min time point in pH 6.8 phosphate buffer (official media).

Tafamidis Tablets 61 mg Prototype Formula Development

Based on outcome of tafamidis meglumine tablets 20 mg, the same strategy was applied for tafamidis tablets 61 mg with increase in tablets weight to study the impact of acidifier in tafamidis tablets 61 mg.

### Example 24. Tafamidis Tablet (Organic Acidifier)

In order to prevent the gel formation of tablets containing Tafamidis 61 mg, it was decided to acidify the blend using acidifier i.e. citric acid anhydrous. The prototype composition is presented in Table 29.

TABLE 29
Compositional makeup of Example 23 (tablet)

| | |
|---|---|
| | Example 24 |
| Ingredient(s) | mg/tablet |

Intra-granular portion

| | |
|---|---|
| Tafamidis | 61.00 |
| Microcrystalline Cellulose (Vivapur PH 101) | 46.80 |
| Lactose monohydrate 200 mesh | 67.20 |
| Croscarmellose sodium (Vivasol) | 5.00 |
| Sodium lauryl sulfate (Stepanol WA-100) | 2.00 |
| Povidone (Kollidon K30) | 5.00 |

Granulating fluid

| | |
|---|---|
| Purified water | qs |
| Citric acid anhydrous | 5.00 |

Extra-granular portion

| | |
|---|---|
| Croscarmellose sodium (Vivasol) | 5.00 |
| Colloidal silicon dioxide (Aerosil 200) | 2.00 |
| Magnesium stearate | 1.00 |
| Total Tablet weight | 200.00 |

Executed batch size-1000 tablets

### Brief Manufacturing Procedure:

Step 1: All the raw materials were dispensed as per batch size quantity.

Step 2: Intragranular portion (tafamidis, microcrystalline cellulose, lactose monohydrate, croscarmellose sodium, sodium lauryl sulfate, povidone) were sifted using #25 mesh and mixed for 10 minutes.

Step 3: Citric acid anhydrous was dissolved in Purified water to provide granulating fluid.

Step 4: Step 2 obtained materials were granulated using Citric acid solution and extra purified water were added until a suitable wet mass were obtained.

Step 5: Wet mass were dried in tray dryer at 60±5° C. until to reach the Loss on drying (LOD) NMT 2.0% w/w.

Step 6: Dried granules were sifted using #25 mesh and retains were milled if required.

Step 7: Croscarmellose sodium (extragranular) was sifted through #25 mesh and blended with step 6 obtained materials for 10 minutes.

Step 8: Step 7 obtained materials were blended with #40 mesh passed colloidal silicon dioxide and Magnesium stearate for 5 minutes.

Step 9: The blend was compressed using suitable tooling with the average tablet weight 200.00 mg.

The compressed tablets were subjected to physical observation in different dissolution media. The observations were presented in below.

TABLE 30a
Dissolution Data (pH 6.8 and 0.1N HCl) for Example 23 (tablets)

| | Dissolution condition: 900 mL, 75 rpm, USP II (Paddle) | | | |
|---|---|---|---|---|
| | pH 6.8 Phosphate buffer | | 0.1N HCl | |
| | | Tablet | | Tablet |
| | Gel | Disintegration/ | Gel | Disintegration/ |
| | formation | Dispersion | formation | Dispersion |
| | Yes/No | time | Yes/No | time |
| Example 23 | No gelation | Within 5 min | No gelation | within 7 min |
| | observed | | observed | |

TABLE 30b
Dissolution Data (pH 4.5 and purified water) for Example 23
(tablets)

| | Dissolution condition: 900 mL, 75 rpm, USP II (Paddle) | | | |
|---|---|---|---|---|
| | pH 4.5 Acetate buffer | | Purified water | |
| | | Tablet | | Tablet |
| | Gel | Disintegration/ | Gel | Disintegration/ |
| | formation | Dispersion | formation | Dispersion |
| | Yes/No | time | Yes/No | time |
| Example 23 | No gelation | Within | No gelation | within 7 min |
| | observed | 10 min | observed | |

TABLE 30c
Dissolution data (official media) for Example 23 (tablets)

| | Official Media: 50 mM | | | | | | | |
|---|---|---|---|---|---|---|---|---|
| | Sodium Phosphate buffer pH 6.8 + 1% | | | | | | | |
| | Tween 80, 900 mL, 75 rpm, USP II (Paddle) | | | | | | | |
| Batch no. | 5 | 10 | 15 | 20 | 30 | 45 | 60 | Inf |
| Example 23 | 36 | 53 | 63 | 68 | 76 | 84 | 88 | 92 |

Based on Table 31 results of tablets in different media, it was observed that in the presence of citric acid anhydrous in tablets prevents the gelation phenomena of tablets containing tafamidis when contacts with water or other dissolution media. A possible reason for disintegration/dispersion of tablets into particles may be due to acidifying the blend using citric acid anhydrous helps to disperse the tablets easily in buffer medias.

### Example 25. Manufacturing Composition and Process of Tafamidis Capsules 61 mg Using TFASSF(16)

Tafamidis (61 mg) Capsules 61 mg manufacturing composition (Ex. 25) and process are presented in Table 31.

TABLE 31
Compositional makeup of Example 25

| | | | |
|---|---|---|---|
| Item | Name of the Ingredient(s) | mg/Capsules | % Percentage |

Intra-granular portion

| | | | |
|---|---|---|---|
| 1. | TFASSF(16) | 72.50 | 36.25 |
| 2. | Povidone (Kollidon K30) | 5.00 | 2.50 |
| 3. | Lactose monohydrate 200 mesh | 102.90 | 51.45 |
| 4. | Croscarmellose sodium (Vivasol) | 10.00 | 5.00 |

Granulating fluid

| | | | |
|---|---|---|---|
| 5. | Purified water | qs | — |
| 6. | Polysorbate 20 | 0.40 | 0.20 |
| 7. | Sodium lauryl sulfate | 1.20 | 0.60 |
| 8. | Fumaric acid | 6.00 | 3.00 |

Extra-granular portion

| | | | |
|---|---|---|---|
| 9. | Colloidal silicon dioxide (Aerosil 200) | 1.00 | 0.50 |
| 10. | Magnesium stearate | 1.00 | 0.50 |

| | | |
|---|---|---|
| Total weight | 200.00 | 100.00 |
| Hard Gelatin Capsule Shell | Size ‘3’ | — |

### Brief Manufacturing Procedure:

Step 1: Dispense all the raw materials.

Step 2: Sift TFASSF(16) (Item 1), Povidone (Item 2), lactose monohydrate (Item 3), Croscarmellose sodium (Item 4), using #18 mesh and mixed for 10 minutes in HSMG.

Step 3: Dissolve Polysorbate 20 (Item 6), Sodium lauryl sulfate (Item 7) and disperse Fumaric acid (Item 8) in Purified water (Item 5).
Step 4: Granulate Step 2 obtained materials using step 3 granulating fluid and add extra purified water until to get a suitable wet mass if required.
Step 5: Dry the wet mass in tray dryer at 60±5° C. until to get a Loss on drying (LOD) of NMT 2.0% w/w.
Step 6: Sift the dried granules using #30 mesh and mill the retains if required.
Step 7: Blend the Step 6 obtained materials with #40 mesh passed colloidal silicon dioxide (Item 9) and Magnesium stearate (Item 10) for 5 minutes in V-Blender.
Step 9: Final blend filled with size 3 capsules with the average weight of 200.00 mg.

The Example 25 Capsules were subjected to physical observation in different dissolution media. The observations are presented in Tables 31a-31c.

TABLE 31a
Dissolution Data (pH 6.8 and 0.1N HCl) for Example 25 (capsule)

| | Dissolution condition: 900 mL, 75 rpm, USP II (Paddle) | | | |
|---|---|---|---|---|
| | pH 6.8 Phosphate buffer | | 0.1N HCl | |
| | | Tablet | | Tablet |
| | Gel | Disintegration/ | Gel | Disintegration/ |
| | formation | Dispersion | formation | Dispersion |
| | Yes/No | time | Yes/No | time |
| Example 25 | No gelation | Within 3 min | No gelation | Within 6 min |
| | observed | | observed | |

TABLE 31b
Dissolution Data (pH 4.5 and water) for Example 25 (capsule)

| | Dissolution condition: 900 mL, 75 rpm, USP II (Paddle) | | | |
|---|---|---|---|---|
| | pH 4.5 Acetate buffer | | Purified water | |
| | | Tablet | | Tablet |
| | Gel | Disintegration/ | Gel | Disintegration/ |
| | formation | Dispersion | formation | Dispersion |
| | Yes/No | time | Yes/No | time |
| Example 25 | No gelation | Within 4 min | No gelation | Within 2 min |
| | observed | | observed | |

TABLE 31c
Dissolution profiling of Tafamidis free acid Capsules 61 mg

| | Official Media: 50 mM | | | | | | | |
|---|---|---|---|---|---|---|---|---|
| | Sodium Phosphate buffer pH 6.8 + 1% | | | | | | | |
| | Tween 80, 900 mL, 75 rpm, USP II (Paddle) | | | | | | | |
| Batch no. | 5 | 10 | 15 | 20 | 30 | 45 | 60 | Inf |
| Example 25 | 42 | 73 | 83 | 87 | 88 | 90 | 91 | 94 |

Inference: Based on above physical behavior of capsules, it was confirmed that in the presence of fumaric acid in capsules would prevents the gelation phenomena of capsules containing TFASSF(16) when contacted with water or other dissolution media (e.g., the official media). The possible reason for disintegration/dispersion of capsules into particles could be due to acidifying the blend using fumaric acid helps to disperse the capsules easily in buffer Medias.

The Example 25 hard gelatin capsule is stable and it shows higher cumulative drug release than WO2016038500A1 (Form 1) and US20210363116A1 (Form CSV) in pH 6.8 PBS compared with soft gelatin capsules including Form 1 (WO2016038500A Form 1 or Form CSV (US20210363116A1). Accordingly, it is concluded that hard gelatin capsule comprising TFASSF(16) has better bioavailability.

### Example 26. Manufacturing Composition and Process of Tafamidis Capsules 61 mg Using TFASSF(16)

Table 32 reports the compositional makeup of tafamidis (61 mg) capsules (Ex. 26) using TFASSF(16).

TABLE 32

| | | | |
|---|---|---|---|
| Item | Name of the Ingredient(s) | mg/Capsules | % Percentage |

Intra-granular portion

| | | | |
|---|---|---|---|
| 1. | TFASSF(16) | 72.50 | 36.25 |
| 2. | Ultraburst | 121.90 | 60.95 |

Granulating fluid

| | | | |
|---|---|---|---|
| 3. | Purified water | qs | — |
| 4. | Polysorbate 20 | 0.40 | 0.20 |
| 5. | Povidone (Kollidon K30) | 3.00 | 1.50 |
| 6. | Sodium lauryl sulfate | 1.20 | 0.60 |

Extra-granular portion

| | | | |
|---|---|---|---|
| 7. | Magnesium stearate | 1.00 | 0.50 |

| | | |
|---|---|---|
| Total weight | 200.00 | 100.00 |
| Hard Gelatin Capsule Shell | Size ‘3’ | — |

### Brief Manufacturing Procedure:

Step 1: Dispense all the raw materials.
Step 2: Sift TFASSF(16) (Item 1) and Ultraburst (Item 2), using #18 mesh and mixed for 10 minutes in HSMG.
Step 3: Dissolve Polysorbate 20 (Item 4), Povidone (Item 5) and Sodium lauryl sulfate (Item 6) in Purified water (Item 3).
Step 4: Granulate Step 2 obtained materials using step 3 granulating fluid and add extra purified water until to get a suitable wet mass if required.
Step 5: Dry the wet mass in tray dryer at 60±5° C. until to get a Loss on drying (LOD) of NMT 2.0% w/w.
Step 6: Sift the dried granules using #30 mesh and mill the retains if required.
Step 7: Blend the Step 6 obtained materials with #40 mesh passed Magnesium stearate (Item 7) for 5 minutes in V-Blender.
Step 9: Final blend filled with size 3 capsules with the average weight of 200.00 mg.

The Example 26 Capsules were subjected to physical observation in different dissolution media. The observations are presented in Tables 32a-32c.

TABLE 32a
Dissolution Data (pH 6.8 and 0.1 N HC1) for Example 26 (capsule)

| | Dissolution condition: 900 mL, 75 rpm, USP II (Paddle) | | | |
|---|---|---|---|---|
| | pH 6.8 Phosphate buffer | | 0.1N HCl | |
| | | Tablet | | Tablet |
| | Gel | Disintegration/ | Gel | Disintegration/ |
| | formation | Dispersion | formation | Dispersion |
| | Yes/No | time | Yes/No | time |
| Example 26 | No gelation | Within 2 min | No gelation | within 2 min |
| | observed | | observed | |

TABLE 32b
Dissolution Data (pH 4.5 and water) for Example 26 (capsule)

| | Dissolution condition: 900 mL, 75 rpm, USP II (Paddle) | | | |
|---|---|---|---|---|
| | pH 4.5 Acetate buffer | | Purified water | |
| | | Tablet | | Tablet |
| | Gel | Disintegration/ | Gel | Disintegration/ |
| | formation | Dispersion | formation | Dispersion |
| | Yes/No | time | Yes/No | time |
| Example 26 | No gelation | Within 3 min | No gelation | within 3 min |
| | observed | | observed | |

TABLE 32c
Dissolution profiling of Tafamidis free acid Capsules 61 mg

| | Official Media: 50 mM | | | | | | | |
|---|---|---|---|---|---|---|---|---|
| | Sodium Phosphate buffer pH 6.8 + 1% | | | | | | | |
| | Tween 80, 900 mL, 75 rpm, USP II (Paddle) | | | | | | | |
| Batch no. | 5 | 10 | 15 | 20 | 30 | 45 | 60 | Inf |
| Example 26 | 49 | 60 | 68 | 73 | 80 | 87 | 91 | 94 |

Inference: Based on above physical behavior of capsules, it was confirmed that in the presence of Ultraburst in capsules would prevent the gelation phenomena of capsules containing TFASSF(16) when contacted with water or other dissolution media (e.g., official media). The possible reason for disintegration/dispersion of capsules into particles could be due to acidifying the blend using Ultraburst helps to disperse the capsules easily in buffer Medias.

The hard gelatin (Example 26) capsule is stable showing higher cumulative drug release than soft gelatin capsule as described in WO2016038500 (Form 1) and US20210363116A1 (Form CSV) in pH 6.8 PBS.

### Example 27. Manufacturing Composition and Process of Tafamidis Capsules 61 mg Using TFASSF(16)

Table 33 reports the compositional makeup of tafamidis (61 mg) capsules (Ex. 27) using TFASSF(16).

TABLE 33
Compositional makeup of Example 27

| | | | |
|---|---|---|---|
| | | mg/ | % |
| Item | Name of the Ingredient(s) | Capsules | Percentage |

Intra-granular portion

| | | | |
|---|---|---|---|
| 1. | TFASSF(16) | 72.500 | 18.125 |
| 2. | Microcrystalline Cellulose 102 | 150.000 | 37.500 |
| 3. | Lactose Anhydrous DT | 149.100 | 37.275 |
| 4. | Croscarmellose sodium (Vivasol) | 10.000 | 2.500 |

Granulating fluid

| | | | |
|---|---|---|---|
| 5. | Purified water | qs | — |
| 6. | Polysorbate 20 | 0.400 | 0.100 |

Extra-granular portion

| | | | |
|---|---|---|---|
| 7. | Croscarmellose sodium (Vivasol) | 10.000 | 2.500 |
| 8. | Colloidal silicon dioxide (Aerosil | 4.000 | 1.000 |
| | 200) | | |
| 9. | Magnesium stearate | 4.000 | 1.000 |

| | | |
|---|---|---|
| Total weight | 400.00 | 100.00 |
| Hard Gelatin Capsule Shell | Size ‘0’ | — |

*Particle size of API: D (0.9): 23.408 μm.

### Brief Manufacturing Procedure:

Step 1: Dispensed all the raw materials.
Step 2: Sifted TFASSF(16) (Item 1), Microcrystalline Cellulose 102 (Item 2), lactose Anhydrous DT (Item 3), Croscarmellose sodium (Item 4), using #18 mesh and mixed for 10 minutes in HSMG.
Step 3: Dissolved Polysorbate 20 (Item 6) in Purified water (Item 5).
Step 4: Granulated Step 2 obtained materials using step 3 granulating fluid and add extra purified water until to get a suitable wet mass if required.
Step 5: Sifted the wet mass using #16 mesh and dry the wet mass in tray dryer at 55±5° C. until to get a Loss on drying (LOD) of NMT 2.0% w/w.
Step 6: Sifted the dried granules using #30 mesh and mill the retains if required.
Step 7: Blended the Step 6 obtained materials with #30 mesh passed Croscarmellose sodium (Item 7) for 10 minutes in V-Blender.
Step 8: Blended the Step 7 obtained materials with #40 mesh passed colloidal silicon dioxide (Item 8) and Magnesium stearate (Item 9) for 5 minutes in V-Blender.
Step 9: Final blend was filled with size “0” capsules with the average weight of 400.00 mg.

### Example 28. Manufacturing Composition & Process of Tafamidis Capsules 61 mg Using TFASSF(16)

Table 34 reports the compositional makeup of tafamidis (61 mg) capsules (Ex. 28) using TFASSF(16) with no surfactant.

TABLE 34
Compositional makeup of Example 28

| | | | |
|---|---|---|---|
| | | mg/ | % |
| Item | Name of the Ingredient(s) | Capsules | Percentage |

Intra-granular portion

| | | | |
|---|---|---|---|
| 1. | TFASSF(16) | 72.500 | 18.125 |
| 2. | Microcrystalline Cellulose 102 | 150.000 | 37.500 |
| 3. | Lactose Anhydrous DT | 149.500 | 37.375 |
| 4. | Croscarmellose sodium (Vivasol) | 10.000 | 2.500 |

Granulating fluid

| | | | |
|---|---|---|---|
| 5. | Purified water | qs | — |

Extra-granular portion

| | | | |
|---|---|---|---|
| 6. | Croscarmellose sodium (Vivasol) | 10.000 | 2.500 |
| 7. | Colloidal silicon dioxide (Aerosil | 4.000 | 1.000 |
| | 200) | | |
| 8. | Magnesium stearate | 4.000 | 1.000 |

| | | |
|---|---|---|
| Total weight | 400.00 | 100.00 |
| Hard Gelatin Capsule Shell | Size ‘0’ | — |

*Particle size of API: D (0.9): 23.408 μm.

### Brief Manufacturing Procedure:

Step 1: Dispensed all the raw materials.
Step 2: Sifted TFASSF(16) (Item 1), Microcrystalline Cellulose 102 (Item 2), lactose anhydrous DT (Item 3), croscarmellose sodium (Item 4), using #18 mesh and mixed for 10 minutes in HSMG.
Step 3: Granulated Step 2 obtained materials using Purified water (Item 5) and add extra purified water until to get a suitable wet mass if required.
Step 5: Sifted the wet mass using #16 mesh and dry the wet mass in tray dryer at 55±5° C. until to get a Loss on drying (LOD) of NMT 2.0% w/w.
Step 6: Sifted the dried granules using #30 mesh and mill the retains if required.
Step 7: Blended the Step 6 obtained materials with #30 mesh passed croscarmellose sodium (Item 6) for 10 minutes in V-Blender.
Step 8: Blended the Step 7 obtained materials with #40 mesh passed colloidal silicon dioxide (Item 7) and Magnesium stearate (Item 8) for 5 minutes in V-Blender.
Step 9: Final blend was filled with size “0” capsules with the average weight of 400.00 mg.

TABLE 35
Dissolution Data for Examples 27-28

| | Dissolution at 50 mM Sodium phosphate buffer pH 6.8 + | | | | | | | | |
|---|---|---|---|---|---|---|---|---|---|
| | 1.0% tween 80, 900 mL, 75 rpm, USP II (Paddle) | | | | | | | | |
| | % drug release | | | | | | | | |
| | 5 | 10 | 15 | 20 | 30 | 45 | 60 | | |
| Example(s) | min | min | min | min | min | min | min | Inf. | Observation |
| Vyndaqel capsule Europe | 0 | 2 | 20 | 63 | 93 | 96 | 97 | 98 | |
| RLD Lot #: 4638510AF | | | | | | | | | |
| Vyndamax capsule USA | 0 | 0 | 7 | 51 | 91 | 95 | 96 | 97 | |
| RLD Lot #: 4815517A | | | | | | | | | |
| Example 27 | 36 | 51 | 60 | 66 | 74 | 81 | 85 | 88 | No rigid |
| Example 27 stored 1 M | 31 | 47 | 56 | 62 | 69 | 76 | 81 | 85 | gel formed |
| at 40° C./75% RH | | | | | | | | | |
| Example 27 stored 1 M | 33 | 49 | 58 | 64 | 72 | 79 | 84 | 87 | |
| at 40° C./75% RH | | | | | | | | | |
| with desiccant | | | | | | | | | |
| Example 28 | 33 | 56 | 68 | 75 | 83 | 89 | 92 | 94 | |
| Example 28 stored 1 M | 48 | 54 | 63 | 69 | 77 | 82 | 85 | 90 | |
| at 40° C./75% RH | | | | | | | | | |
| Example 28 stored 1 M | 45 | 54 | 63 | 68 | 74 | 79 | 83 | 89 | |
| at 40° C./75% RH | | | | | | | | | |
| with desiccant | | | | | | | | | |

*1 M—1 month; RH—Relative humidity Inf—Infinity

### Observation:

The RLD compositions in Vyndagel and Vyndamax are soft gelatin capsule having suspension of Tafamidis meglumine and tafamidis free acid respectively. Based on the above table it is confirmed that the dissolution of profile of present oral solid formulation is comparable to the RLD composition and there is no formation of rigid gel during the dissolution at official media containing 50 mM Sodium phosphate buffer pH 6.8+1.0% tween 80, 900 mL, 75 rpm, USP II (Paddle).

### Example 29. Bioequivalence Study

A bioequivalence study was conducted with 24 subjects (healthy human adult men, mean age of 33.6 y (range: 22-42 y)), all 24 subjects completed the study performed using the Example 27 and Example 28 capsule formulation. The bioequivalence study included an open-label, balanced, randomized, single-dose, two-treatment, two-sequence, two-period, two-way crossover, in healthy adult human subjects under fed conditions and the results are compared with available reference listed drug (RLD, Vyndamax (tafamidis (61 mg)) capsules). Table 36 reports the mean pharmacokinetic parameters and the observed standard deviation (SD), as well as the % coefficient of variation for Cmax- and AUC-values and the range (minimum and maximum values) for Tmax.

TABLE 36
Bioequivalence study data for drug product of Examples 27-28 with
reference product

| Treatments (Dose, | Mean Parameters ± SD (% CV/Range) | | |
|---|---|---|---|
| Dosage Form, Route) | | AUC₀₋₇₂ (hr* | |
| [Product ID] | Cₘₐₓ (μg/mL) | μg/mL) | Tₘₐₓ (hr) |
| Reference Product (R): | 4.431 ± 0.8755 | 154.197 ± 24.1827 | 4.50 |
| Vyndamax ™ (tafamidis) | (19.76) | (15.68) | (1.00-12.02) |
| capsules 61 mg | | | |
| [Lot # 4815517A] | | | |
| Test Product (T1): | 4.514 ± 1.2674 | 152.267 ± 34.4707 | 4.76 |
| Tafamidis capsules 61 | (28.08) | (22.64) | (2.50-24.00) |
| mg [Example 27] | | | |
| Test Product (T2): | 4.727 ± 0.9390 | 169.582 ± 30.7156 | 4.50 |
| Tafamidis capsules 61 | (19.86) | (18.11) | (2.00-12.00) |
| mg [Example 28] | | | |

Based on above bioequivalence study it is confirmed that the exemplified tafamidis capsule (Example 27 with surfactant and Example 28 without surfactant) obtained as disclosed herein are bioequivalent to the reference product (Vyndamax (tafamidis, 61 mg) capsules).

### CITED INFORMATION

Dissolution Medium for tafamidis/tafamidis meglumine, as of Nov. 12, 2021.
International Publication No. WO 2016/038500 A1, Crystalline solid forms of 6-carboxy-2-(3,5-dichlorophenyl)-benzoxazole, to Girard et al. of Pfizer Inc. (“Girard”).
International Publication No. WO 2020/128816 A2, Pharmaceutical compositions and methods comprising a combination of a benzoxazole transthyretin stabilizer and an additional therapeutic agent, to Bulawa et al. of Pfizer Inc. (“Bulawa”).
International Publication No. WO 2020/232325 A1, Solid state forms of tafamidis and salts thereof to Matečić et al. of Teva Pharmaceuticals International GmbH (“Matečić”).
International Publication No. WO 2021/001858 A1, Improved process for the preparation of 2-(3,5-dichlorophenyl-1,3-benzoxazole-6-carboxylic acid or its pharmaceutically acceptable salts and polymorphs thereof, to Sajja et al. of MSN Laboratories Private Limited, R&D Center (“Sajja”).
International Publication No. WO 2021/019448 A1, Process for the preparation of transthyretin dissociation inhibitor, to Rathnakar et al. of Honour (R&D) (“Rathnakar”).
The United States Pharmacopeia (USP 33)|The National Formulary (NF 28), reissue (2010), Chapter <711>, Dissolution.
U.S. Patent Application Publication No. 2014/0377185 A9, Stabilized formulations containing iodinated contrast agents and cyclodextrins, to Mosher, G. L. of Verrow Pharmaceuticals, Inc. (“Mosher”).
U.S. Patent Application Publication No. US 2021/0363116 A1, Crystal form of tafamidis and preparation method therefor and use thereof, to Chen et al. of Crystal Pharmaceutical (Suzhou) Co., Ltd. (“Chen”).
Vyndaqel® (tafamidis meglumine) capsules/Vyndamax® (tafamidis) capsules Prescribing Information, as of Jun. 1, 2021.

Vyndaqel® European Public assessment report (Sep. 21, 2011).

The subject matter of U.S. patent application Ser. No. 17/530,003 (filed on Nov. 18, 2021) and Ser. No. 17/863,754 (filed on Jul. 13, 2022) is hereby incorporated by reference in its entirety. Additionally, the subject matter of the documents cited herein is incorporated by reference in their entirety to the extent necessary. If there is a difference in meaning between the incorporated terms and the terms disclosed herein, the meaning of the terms disclosed herein will control.

Those skilled in the art will also appreciate that various adaptations and modifications of the preferred and alternative embodiments described above can be configured without departing from the scope and spirit of the disclosure. Therefore, it is to be understood that, within the scope of the appended claims, the disclosure may be practiced other than as specifically described herein.

1. A solid oral dosage form, comprising:
a) tafamidis or a pharmaceutically acceptable salt or its solid-state forms or polymorphic forms thereof;
b) at least one acidifier in an amount of about 0% w/w to about 10% w/w based on the total weight of the dosage form; and
c) at least one pharmaceutically acceptable excipient.

2. The solid oral dosage form of claim 1, wherein the solid oral dosage form comprises a hard gelatin capsule, an HPMC capsule, or a tablet.

3. The solid oral dosage form of claim 1 comprising 20 mg of tafamidis meglumine.

4. The solid oral dosage form of claim 1, wherein the solid dosage form comprises about 61 mg of tafamidis.

5. The solid oral dosage form of claim 1, wherein the solid dosage form comprises about 12.2 mg of tafamidis.

6. The solid oral dosage form of claim 1 comprising a solid-state form comprising tafamidis and fumaric acid wherein the solid-state form comprises about 61 mg of tafamidis and fumaric acid in an amount of from about 14% w/w to about 18% w/w based on the total weight of the solid-state form.

7. The solid oral dosage form of claim 1 comprising easily dispersible granules optionally prepared by a process comprising wet granulation or top-spray granulation.

8. The solid oral dosage form of claim 1 comprising easily dispersible granules capable of dispersing without gel formation within about 5 minutes to about 7 minutes in an aqueous medium having a volume of about 900 mL comprising 50 mM sodium phosphate, optionally 1% Tween 80, and water at a pH of 6.8 when stirred at 75 rpm according to USP II Paddle.

9. The solid oral dosage form of claim 1, wherein the at least one pharmaceutically acceptable excipient comprises a disintegrant, a binder, a diluent, a glidant, a lubricant, or a combination thereof.

10. The solid oral dosage form of claim 1, wherein the at least one pharmaceutically acceptable excipient comprises a disintegrant comprising crospovidone, croscarmellose sodium, low hydroxypropyl cellulose, starch, sodium starch glycolate, microcrystalline cellulose, alginic acid, polacrillin potassium, or a combination thereof in an amount of from about 0% w/w to about 10% w/w based on the total weight of the composition.

11. The solid oral dosage form of claim 1, wherein the at least one pharmaceutically acceptable excipient comprises a binder comprising povidone, starch, gelatin, hydroxypropyl cellulose, hydroxypropyl methylcellulose, methyl cellulose, carboxymethyl cellulose, or a combination thereof.

12. The solid oral dosage form of claim 1, wherein the at least one pharmaceutically acceptable excipient comprises a glidant comprising colloidal silicon dioxide, magnesium trisilicate, starch, talc, or a combination thereof in an amount of from about 0% w/w to about 2% w/w based on the total weight of the composition.

13. The solid oral dosage form of claim 1, wherein the at least one pharmaceutically acceptable excipient comprising a lubricant comprising calcium stearate, glycerin monostearate, glyceryl behenate, glyceryl palmitostearate, hydrogenated castor oil, hydrogenated vegetable oil type I, light mineral oil, magnesium lauryl sulfate, magnesium stearate, medium-chain triglycerides, mineral oil, myristic acid, palmitic acid, poloxamer, polyethylene glycol, sodium stearyl fumarate, stearic acid, talc, zinc stearate, or a combination thereof in an amount of from about 0% w/w to about 2% w/w based on the total weight of the composition.

14. The solid oral dosage form of claim 1, wherein the at least one pharmaceutically acceptable excipient comprises a diluent comprising lactose, microcrystalline cellulose, starch, dicalcium phosphate, mannitol, xylitol, sorbitol, dextrose, fructose, sucrose, maltodextrin, or a combination thereof.

15. The solid oral dosage form of claim 1, wherein the at least one pharmaceutically acceptable excipient comprises a diluent in an amount of from about 0% w/w to about 90% w/w based on the total weight of the composition.

16. The solid oral dosage form of claim 1, wherein the at least one pharmaceutically acceptable excipient comprises a diluent in an amount of from about 0% w/w to about 90% w/w based on the total weight of the composition.

17. The solid oral dosage form of claim 1 comprising a hard gelatin composition or a HPMC capsule composition comprising 20 mg of tafamidis meglumine, 61 mg of tafamidis free acid, 12.2 mg of tafamidis free acid, or 72.5 mg of TFASSF (16).

18. The solid oral dosage form of claim 1 comprising a tablet composition comprising 20 mg of tafamidis meglumine, 61 mg of tafamidis free acid, 12.2 mg of tafamidis free acid, or 72.5 mg of TFASSF(16).

19. The solid oral dosage form of claim 1, wherein or a pharmaceutically acceptable salt or its solid-state forms or polymorphic forms thereof has a D90 particle size of from about 20 μm to about 100 μm.

20. The solid oral dosage form of claim 1 that does not include a surfactant.

21. A method of treating transthyretin amyloid disease in a mammal, comprising administering to the mammal the solid oral dosage form of claim 1.

22. The solid oral dosage form, comprising:
a) a solid-state form comprising tafamidis and fumaric acid;
b) at least one acidifier in an amount of about 0% w/w to about 10% w/w based on the total weight of the dosage form; and
c) at least one pharmaceutically acceptable excipient;
wherein the solid-state form comprises fumaric acid in an amount of from about 14% w/w to about 18% w/w based on the total weight of the solid-state form.

23. The solid oral dosage form of claim 22, wherein the solid-state form comprises fumaric acid in an amount of from about 15.0% w/w to about 17.0% w/w based on the total weight of the solid-state form.

24. The solid oral dosage form of claim 22, wherein the solid-state form comprises fumaric acid in an amount of from about 15.9% w/w to about 16.2% w/w based on the total weight of the solid-state form.

25. The solid oral dosage form of claim 22, wherein the solid-state form comprises fumaric acid in an amount of in an amount of about 16% w/w based on the total weight of the solid-state form.

26. The solid oral dosage form of claim 22, wherein the solid dosage form comprises about 61 mg of tafamidis.

27. The solid oral dosage form of claim 22, wherein the solid dosage form comprises about 12.2 mg of tafamidis.

28. A method of treating transthyretin amyloid disease in a mammal, comprising administering to the mammal the solid oral dosage form of claim 22.
